(19) United States
(12) Patent Application Publication
(10) Pub. No.: US 2022/0378461 A1
(43) Pub. Date: Dec. 1, 2022

(54) THERAPEUTIC ULTRASOUND TISSUE TREATMENT SYSTEMS, APPARATUSES, AND METHODS

(71) Applicant: Otsuka Medical Devices Co., Ltd., Tokyo (JP)

(72) Inventors: Desmond Cheung, San Jose, CA (US); Shruthi Thirumalai, Fremont, CA (US); Liang Zhai, Belmont, CA (US); James D. Mazzone, San Jose, CA (US); Jaime MERINO, Mountain View, CA (US); Eric Dailey, San Jose, CA (US); Lewis J. Thomas, La Paz (MX)

(21) Appl. No.: 17/817,540

(22) Filed: Aug. 4, 2022

Related U.S. Application Data
(63) Continuation-in-part of application No. 17/702,633, filed on Mar. 23, 2022.
(60) Provisional application No. 63/303,261, filed on Jan. 26, 2022.
(60) Provisional application No. 63/164,986, filed on Mar. 23, 2021.
(60) Provisional application No. 63/303,261, filed on Jan. 26, 2022.

(51) Int. Cl. A61B 17/32; B06B 1/06

(52) U.S. Cl. CPC A61B 17/320068; B06B 1/0655; A61B 2017/320069; A61B 2017/00402

(57) ABSTRACT

A tissue treatment catheter and system include a catheter shaft sized and shaped for delivery through a radial artery to a blood vessel of a patient. The catheter shaft has several lumens, including a guidewire lumen, a cable lumen, and one or more fluid lumens. A stiffening web extends from the guidewire lumen and is thicker than an outer wall of the catheter shaft. The tissue treatment catheter and system include an ultrasound transducer, a balloon surrounding the ultrasound transducer, and a single electrical cable electrically connected to the ultrasound transducer to deliver sufficient electrical energy during sonication to the transducer such that the transducer thermally induces modulation of neural fibers surrounding the blood vessel sufficient to improve a measurable physiological parameter corresponding to a diagnosed condition of the patient. Other embodiments are described and claimed.

### PRIORITY CLAIM

This application is a continuation-in-part of and claims priority to U.S. patent application Ser. No. 17/702,633, filed Mar. 23, 2022, which claims the benefit of priority under 35 U.S.C. § 119(e) to U.S. Provisional Patent Application No. 63/164,986, filed Mar. 23, 2021, and U.S. Provisional Patent Application No. 63/303,261, filed Jan. 26, 2022. This application claims priority to U.S. Provisional Patent Application No. 63/303,261, filed Jan. 26, 2022. Each of the foregoing applications are incorporated herein by reference to provide continuity of disclosure.

### FIELD OF THE TECHNOLOGY

This application relates generally to minimally-invasive apparatuses, systems and methods that provide energy delivery to a targeted anatomical location of a subject, and more specifically, to catheter-based, intraluminal apparatuses, systems and methods including or utilizing an ultrasound transducer configured to emit ultrasonic energy for the treatment of tissue, such as nerve tissue.

### BACKGROUND

According to the Centers for Disease Control and Prevention (CDC), about one in every three adults suffer from high blood pressure, also known as hypertension. Left untreated, hypertension can result in renal disease, arrhythmias, and heart failure. In recent years, the treatment of hypertension has focused on minimally invasive interventional approaches to inactivate the renal nerves surrounding the renal artery. Autonomic nerves tend to follow blood vessels to the organs that they enervate. Catheters may reach specific structure that may be proximate to the lumens in which they travel. For example, one system employs a radiofrequency (RF) generator connected to a catheter having multiple electrodes placed against the intima of the renal artery and used to create an electrical field in the vessel wall and surrounding tissue that results in resistive (ohmic) heating of the tissue to a temperature sufficient to ablate the tissue and the renal nerve passing through that tissue. To treat all the renal nerves surrounding the renal arteries, the RF electrodes are repositioned several times around the inside of the renal artery. However, the relatively confined electric fields created by the RF electrodes may miss some of the renal nerves, leading to an incomplete treatment. Additionally, to heat the renal nerves, the RF electrodes must contact the intima, posing a risk of damage or necrosis to the intima, which in turn can lead to thrombus formation, fibrosis of the vessel wall, mechanical weakening of the vessel and possible vessel dissection.

U.S. Pat. Nos. 9,943,666, 9,981,108, and 10,039,901 to Warnking, U.S. Pat. Nos. 9,700,372, 9,707,034, and 10,368,944 to Schaer, and U.S. Pat. Nos. 10,350,440 and 10,456,605 to Taylor, the entire contents of each which is incorporated by reference herein, solve many of the drawbacks of a RF system such as described above. An example embodiment of the system includes an ultrasound transducer positioned along a distal end of a catheter designed to be inserted into a blood vessel (e.g., the renal artery). Electrical cabling, which is received within a cabling lumen of the catheter, can be used to power the ultrasound transducer. The ultrasound transducer emits one or more therapeutic doses of unfocused ultrasound energy, which heats the tissue adjacent to the body lumen within which the transducer is disposed. Such unfocused ultrasound energy may, for example, ablate target nerves surrounding that body lumen, but without damaging non-target tissue such as the inner lining of the body lumen or unintended organs outside of the body lumen. The system may include a balloon mounted at the distal end of the catheter that is designed to cool the blood vessel when a cooling fluid is delivered to the balloon. Such a design enables creation of one or more ablation zones sufficient to achieve long-term nerve inactivation at different locations around the circumference of the blood vessel.

The ultrasound transducer may include first and second electrodes which are arranged on either side of a cylindrical piezoelectric material, such as lead zirconate titanate (PZT). To energize the transducer, a voltage is applied across the first and the second electrodes at frequencies selected to cause the piezoelectric material to resonate, thereby generating vibration energy that is emitted radially outward from the transducer. The transducer is designed to provide a generally uniform and predictable emission profile, to inhibit damage to surrounding non-target tissue. In addition, a cooling fluid is circulated through the balloon, both prior to, during, and after activation of the transducer, so as to reduce heating of an inner lining of the body lumen and to cool the transducer. In this manner, the peak temperatures achieved by tissue within the cooling zone remain lower than for tissue located outside the cooling zone.

### SUMMARY

A system, according to an embodiment of the present technology, comprises a catheter sized and shaped for delivery through a radial artery to a blood vessel of a patient, wherein the catheter comprises: a catheter shaft having a distal end and a proximal end, a plurality of lumens extending longitudinally through the catheter shaft between the distal end and the proximal end thereof, an ultrasound transducer distally positioned relative to the distal end of the catheter shaft, the ultrasound transducer including a piezoelectric transducer body and first and second electrodes that are electrically isolated from one another, a balloon surrounding the ultrasound transducer, wherein at least one of the plurality of lumens is configured to provide a cooling fluid to the balloon at a pressure and flow rate sufficient to protect non-target tissue in the blood vessel wall from thermal injury, a single electrical cable electrically connected to the ultrasound transducer, the single electrical cable comprising one or more first conductor(s) coupled to the first electrode of the ultrasound transducer, and one or more second conductor(s) coupled to the second electrode of the ultrasound transducer, wherein the single electrical cable is configured to deliver sufficient electrical energy during sonication to the transducer such that the transducer thermally induces modulation of neural fibers surrounding the blood vessel sufficient to improve a measurable physiological parameter corresponding to a diagnosed condition of the patient.

The one or more first conductor(s) of the single electrical cable may comprise a pair of inner conductors that are both electrically coupled to the first electrode of the ultrasound transducer, and the one or more second conductor(s) of the single electrical cable may comprise an outer tubular conducting shield that surrounds the pair of inner conductors, the outer tubular conducting shield including a plurality of conductors that are bundled together and electrically coupled to the second electrode of the ultrasound transducer.

At least one of the plurality of lumens may be a cable lumen and the single electrical cable may extend through the cable lumen. In certain embodiments, the catheter shaft surrounds the electrical cable such that no gap is between a lumen wall of the cable lumen and an outer surface of the electrical cable. In an embodiment, each inner conductor of the pair of inner conductors comprises a solid wire. In an embodiment, each inner conductor of the pair of inner conductors, comprises a stranded and twisted wire.

In an embodiment, the piezoelectric transducer body may comprise a hollow tube of piezoelectric material having an inner surface and an outer surface, the first electrode, to which the pair of inner conductors that are both electrically coupled, may be disposed on the outer surface of the hollow tube of piezoelectric material, and the second electrode, to which the bundled together plurality of conductors of the outer tubular conducting shield are electrically coupled, may disposed on the inner surface of the hollow tube of piezoelectric material.

In an embodiment, the piezoelectric transducer body comprises a hollow tube of piezoelectric material having an inner surface and an outer surface, the first electrode is disposed on one of the inner and the outer surfaces of the hollow tube of piezoelectric material, and the second electrode is disposed on the other one of the inner and the outer surfaces of the hollow tube of piezoelectric material.

In an embodiment, the one or more first conductor(s) of the single electrical cable comprises a pair of solid wires that are both electrically coupled to the first electrode of the ultrasound transducer, and the one or more second conductor(s) of the single electrical cable comprises a stranded and twisted wire conductor including a plurality of electrically conductive strands that are bundled together and electrically coupled to the second electrode of the ultrasound transducer.

In an embodiment, the one or more first conductor(s) of the single electrical cable comprises a pair of solid wires that are both electrically coupled to the first electrode of the ultrasound transducer, and the one or more second conductor(s) of the single electrical cable comprises a stranded wire conductor including a plurality of electrically conductive strands that are bundled together and electrically coupled to the second electrode of the ultrasound transducer.

In an embodiment, the piezoelectric transducer body comprises a hollow tube of piezoelectric material having an inner surface and an outer surface, the first electrode, to which the pair of solid wires are both electrically coupled, is disposed on the outer surface of the hollow tube of piezoelectric material, and the second electrode, to which the bundled together plurality of electrically conductive strands of the stranded wire conductor are electrically coupled, is disposed on the inner surface of the hollow tube of piezoelectric material.

In an embodiment, the plurality of lumens consists of four lumens, which include first, second, third and fourth lumens, the first lumen is configured to accept a guidewire; the second lumen is configured to provide the cooling fluid to the balloon, the third lumen is configured to remove the cooling fluid from the balloon, and the fourth lumen is configured to accept the single electrical cable.

In an embodiment, the plurality of lumens consists of three lumens, which include first, second and third lumens, the first lumen is configured to accept a guidewire, the second lumen is configured to provide the cooling fluid to the balloon, the third lumen is configured to remove the cooling fluid from the balloon, and the single electrical cable is mounted within one of the second and the third lumens, and thus, is mounted within a same one of the lumens that is either configured to supply the cooling fluid to the balloon or to remove the cooling fluid from the balloon.

In an embodiment, the one of the second and the third lumens, which the single electrical cable is mounted within, has a greater interior cross-sectional area and a greater interior volume than the other one of the second and the third lumens that is devoid of the single electrical cable. In an embodiment, the one of the second and the third lumens in which the single electrical cable is mounted, has the greater interior cross-sectional area and the greater interior volume than the other one of the second and the third lumens that is devoid of the single electrical cable, so that an available interior cross-sectional area and an available interior volume of the one of the second and the third lumens that receives the single electrical cable, which are available for providing the cooling fluid to the balloon or removing the cooling fluid from the balloon, are respectively substantially the same as an interior cross-sectional area and an interior volume of the other one of the second and the third lumens that is devoid of the single electrical cable. In an embodiment, an outer diameter of the catheter shaft is within a range of 0.04 inches to 0.055 inches, inclusive and the working length of the catheter shaft is at least 145 cm. In certain embodiments, an outer diameter of the catheter shaft is within a range of 0.05 inch to 0.07 inch, inclusive and the working length of the catheter shaft is at least 145 cm.

In an embodiment, an outer diameter the catheter has a French gauge of 5 or less. In an embodiment, an outer diameter the transducer has a French gauge of 4, the transducer has a frequency of between 6 MHz and 20 MHz, e.g., 8.5 MHz to 9.5 MHz, 10 MHz, 12 MHz, or 15 MHz and the catheter shaft 214 has an on outer diameter of about 0.05 inch to 0.07 inch and a working length of at least 145 cm.

In an embodiment, the transducer is a water-backed or an air-backed transducer having an outer diameter of French gauge of 4, the transducer has a frequency of between 16 MHz and 20 MHz, e.g., 8.5 MHz to 9.5 MHz, 10 MHz, 12 MHz, or 15 MHz, and the catheter shaft 214 has an on outer diameter of about 0.05 inch to 0.07 inch and a working length of at least 155 cm.

In an embodiment, a characteristic impedance of the single electrical cable is matched to a characteristic impedance of the ultrasound transducer and to a characteristic output impedance of a signal generator to which the single electrical cable is configured to be coupled.

In an embodiment, the single electrical cable is integrated into the catheter shaft and a separate cable lumen may be omitted from the catheter.

In an embodiment, the diagnosed condition of the patient is hypertension and the single electrical cable is configured to deliver at least 30 W to the transducer, such as to provide sufficient power to denervate nerve surrounding the blood vessel to provide a therapeutically beneficial reduction in blood pressure of the patient.

In an embodiment, the cable is configured to deliver 30 W to 50 W to the transducer, such as to provide sufficient power to denervate nerve surrounding one or more blood vessels to significantly improve the patient's blood pressure such as to provide a therapeutically beneficial reduction in blood pressure of the patient.

In an embodiment, the cable is configured to deliver 30 W to 50 W to the transducer, such as to provide sufficient power to denervate nerve surrounding one or more blood vessels in a manner that affects a measurable physiological parameter associated with at least one of hypertension, heart failure, kidney disease, chronic renal failure, sympathetic hyperactivity, diabetes, insulin resistance, fatty liver disease, autoimmune disorders, sepsis, sleep apnea, sleep disorders, bowel and bladder disfunctions, gut motility disorders, urinary urgency, pulmonary hypertension, cancer, metabolic disorder, pancreatic pain, anxiety, depression, eating disorders, Loin Pain Hematuria Syndrome (LPHS), post-traumatic stress disorder, contrast nephropathy, arrhythmia, atrial fibrillation, polycystic kidney disease, autosomal dominant polycystic kidney disease (ADPKD), pain related to polycystic kidney disease, acute myocardial infarction, or cardio-renal syndrome.

In an embodiment, the cable is configured to deliver 30 W to 50 W to the transducer, such as to provide sufficient power to denervate nerve surrounding one or more blood vessels to significantly improve a patient's blood pressure, insulin resistance, diabetic control, renin levels, renal catecholamine (e.g., norepinephrine) spillover, metanephrine, anxiety score, depression score, eating disorder risk, QoL and stress levels, heat shock protein (HSP) expression level for one or more HSPs at or near the target site, urinary sodium excretion, cytokine levels, vascular resistance, pulse pressure, ejection fraction, pulse wave velocity, frequency of arrhythmia, renal function, heart rate, arterial stiffness, urinary potassium excretion, urine catecholamines, pain level, central sympathetic overactivity, size of one or more kidney cysts, AF, congestion, lipid profile, liver enzymes, blood sugar, liver function, markers of inflammation, and/or hemoglobin A1C level.

In an embodiment, the cable is configured to deliver at least 40 W to the transducer, such as to provide sufficient power to denervate nerve surrounding the blood vessel to significantly improve the patient's blood pressure and/or other measurable physiological parameter associated with a diagnosed disease.

In an embodiment, the cable is configured to deliver an energy level of 30 W to 50 W to the transducer for 7 to 10 seconds, such as to provide sufficient power to denervate nerve surrounding the blood vessel to significantly improve the patient's blood pressure and/or other measurable physiological parameter associated with a diagnosed disease, such as to have a therapeutic benefit to the patient.

In an embodiment, the treatment time is 7 seconds.

In an embodiment, the cross-sectional area of the cable is less than or equal to about 0.00022 in².

In an embodiment, the at least one of the plurality of lumens is configured to provide the cooling fluid to the balloon has a cross-sectional area of between about 0.0006 cm² to 0.0008 cm².

In an embodiment, the at least one of the plurality of lumens is configured to maintain a pressure of the cooling fluid at 10 to 30 psi and a 15 to 45 ml/min.

A tissue treatment apparatus, according to an embodiment of the present technology, comprises a catheter including a catheter shaft having a distal end and a proximal end. A plurality of lumens extend longitudinally through the catheter shaft between the distal end and the proximal end thereof. An ultrasound transducer is distally positioned relative to the distal end of the catheter shaft. The ultrasound transducer includes a piezoelectric transducer body and first and second electrodes that are electrically isolated from one another. A single electrical cable extends through one of the plurality of lumens. The single electrical cable includes one or more first conductor(s) coupled to the first electrode of the ultrasound transducer, and one or more second conductor(s) coupled to the second electrode of the ultrasound transducer. The single electrical cable is used to apply a voltage between the first and the second electrodes to thereby cause the piezoelectric transducer body to generate ultrasonic waves. In accordance with certain embodiments, the ultrasonic waves that are generated by the piezoelectric transducer body are used to ablate tissue adjacent to a body lumen into which the ultrasound transducer is inserted.

In accordance with certain embodiments, the one or more first conductor(s) of the single electrical cable comprises a pair of inner conductors that are both electrically coupled to the first electrode of the ultrasound transducer, and the one or more second conductor(s) of the single electrical cable comprises an outer tubular conducting shield that surrounds the pair of inner conductors, the outer tubular conducting shield including a plurality of conductors that are bundled together and electrically coupled to the second electrode of the ultrasound transducer.

In accordance with certain embodiments, each inner conductor, of the pair of inner conductors, comprises a solid wire. In accordance with alternative embodiments, each inner conductor, of the pair of inner conductors, comprises a stranded wire.

In accordance with certain embodiments, the piezoelectric transducer body comprises a hollow tube of piezoelectric material having an inner surface and an outer surface. The first electrode, to which the pair of inner conductors that are both electrically coupled, is disposed on the outer surface of the hollow tube of piezoelectric material. The second electrode, to which the bundled together plurality of conductors of the outer tubular conducting shield are electrically coupled, is disposed on the inner surface of the hollow tube of piezoelectric material.

In accordance with certain embodiments, the one or more first conductor(s) of the single electrical cable comprises a pair of solid wires that are both electrically coupled to the first electrode of the ultrasound transducer, and the one or more second conductor(s) of the single electrical cable comprises a stranded wire conductor including a plurality of electrically conductive strands that are bundled together and electrically coupled to the second electrode of the ultrasound transducer.

In accordance with certain embodiments, a balloon surrounds the ultrasound transducer and is configured to be at least partially filled with a cooling fluid that is supplied to the balloon via one of the lumens and is removed from the balloon via another one of the lumens.

In accordance with certain embodiments, the plurality of lumens consist of four lumens, which include first, second, third and fourth lumens, wherein the first lumen is configured to accept a guidewire, the second lumen is configured to provide the cooling fluid to the balloon, the third lumen is configured to remove the cooling fluid from the balloon, and the fourth lumen is configured to accept the single electrical cable.

Alternatively, in accordance with certain embodiments, the plurality of lumens consist of three lumens, which include first, second and third lumens, wherein the first lumen is configured to accept a guidewire, the second lumen is configured to provide the cooling fluid to the balloon, the third lumen is configured to remove the cooling fluid from the balloon, and the single electrical cable is received within one of the second and the third lumens, and thus, is received within a same one of the lumens that is either configured to supply the cooling fluid to the balloon or to remove the cooling fluid from the balloon. In certain such embodiments, the one of the second and the third lumens, which receives the single electrical cable, has a greater interior cross-sectional area and a greater interior volume than the other one of the second and the third lumens that is devoid of the single electrical cable.

In certain such embodiments, the one of the second and the third lumens that receives the single electrical cable, has the greater interior cross-sectional area and the greater interior volume than the other one of the second and the third lumens that is devoid of the single electrical cable, so that an available interior cross-sectional area and an available interior volume of the one of the second and the third lumens that receives the single electrical cable, which are available for providing the cooling fluid to the balloon or removing the cooling fluid from the balloon, are respectively substantially the same as an interior cross-sectional area and an interior volume of the other one of the second and the third lumens that is devoid of the single electrical cable.

In accordance with certain embodiments, an outer diameter of the catheter, i.e., the larger of the catheter shaft and transducer, is within a range of 4 French gauge to 5 French gauge, inclusive, or more generally, an outer diameter of the catheter has a French gauge of 5 or less.

In accordance with certain embodiments, a characteristic impedance of the single electrical cable is matched to a characteristic impedance of the ultrasound transducer and to a characteristic output impedance of a signal generator to which the single electrical cable is configured to be coupled.

In accordance with certain embodiments, a tissue treatment system comprises a controller including a signal generator, and a catheter including a catheter shaft having a distal end and a proximal end. A plurality of lumens extend longitudinally through the catheter shaft between the distal end and the proximal end thereof. An ultrasound transducer is distally positioned relative to the distal end of the catheter shaft, the ultrasound transducer including a piezoelectric transducer body and first and second electrodes that are electrically isolated from one another. A single electrical cable extends through one of the plurality of lumens. The single electrical cable includes one or more first conductor(s) coupled to the first electrode of the ultrasound transducer, and one or more second conductor(s) coupled to the second electrode of the ultrasound transducer. The single electrical cable is coupled to the signal generator and is used to apply a voltage between the first and the second electrodes to thereby cause the piezoelectric transducer body to generate ultrasonic waves.

In accordance with certain embodiments, a characteristic impedance of the single electrical cable is matched to a characteristic impedance of the ultrasound transducer and to a characteristic output impedance of the signal generator to which the single electrical cable is configured to be coupled.

In accordance with certain embodiments, the ultrasonic waves that are generated by the piezoelectric transducer body are used to ablate tissue adjacent to a body lumen into which the ultrasound transducer is inserted.

In accordance with certain embodiments, the one or more first conductor(s) of the single electrical cable comprises a pair of inner conductors that are both electrically coupled to the first electrode of the ultrasound transducer. The one or more second conductor(s) of the single electrical cable comprises an outer tubular conducting shield that surrounds the pair of inner conductor, wherein the outer tubular conducting shield includes a plurality of conductors that are bundled together and electrically coupled to the second electrode of the ultrasound transducer.

In accordance with certain embodiments, the piezoelectric transducer body comprises a hollow tube of piezoelectric material having an inner surface and an outer surface. The first electrode, to which the pair of inner conductors that are both electrically coupled, is disposed on the outer surface of the hollow tube of piezoelectric material. The second electrode, to which the bundled together plurality of conductors of the outer tubular conducting shield are electrically coupled, is disposed on the inner surface of the hollow tube of piezoelectric material.

A method, according to an embodiment, is for use by a tissue treatment apparatus that comprises a catheter including a catheter shaft having a distal end and a proximal end, a plurality of lumens extending longitudinally through the catheter shaft between the distal end and the proximal end thereof, and an ultrasound transducer distally positioned relative to the distal end of the catheter shaft. The ultrasound transducer includes a piezoelectric transducer body and first and second electrodes that are electrically isolated from one another. The method comprises extending through one of the plurality of lumens, a single electrical cable including one or more first conductor(s) coupled to the first electrode of the ultrasound transducer, and one or more second conductor(s) coupled to the second electrode of the ultrasound transducer. The method also includes applying a voltage between the first and the second electrodes, using the single electrical cable, to thereby cause the piezoelectric transducer body to generate ultrasonic waves.

In accordance with certain embodiments, a balloon surrounds the ultrasound transducer, and the plurality of lumens consist of three lumens, including a first lumen configured to accept a guidewire, a second lumen configured to provide a cooling fluid to the balloon, and a third lumen configured to remove the cooling fluid from the balloon. The single electrical cable extends through one of the second and the third lumens. In certain such embodiments, the method further comprises transferring the cooling fluid between a reservoir and the balloon using the second and the third lumens of the catheter shaft to thereby cool the ultrasound transducer that is surrounded by the balloon, and also cool at least a portion of the single electrical cable that extends through one of the first and the second lumens. In accordance with certain embodiments, the method also comprises ablating tissue, adjacent to a body lumen into which the ultrasound transducer is inserted, using the ultrasonic waves that are generated by the piezoelectric transducer body.

In accordance with certain embodiments, a tissue treatment catheter comprises a catheter shaft having an outer wall sized and shaped for delivery through a radial artery to a blood vessel of a patient. The catheter shaft has several lumens extending longitudinally through the catheter shaft between a distal end and a proximal end. The several lumens include a guidewire lumen within a guidewire lumen wall, and a cable lumen. The catheter shaft includes a stiffening web extending from the guidewire lumen wall. The several lumens include a first fluid lumen defined between the outer wall, the guidewire lumen wall, and the stiffening web. An outer wall thickness of the outer wall is less than a stiffening wall thickness of the stiffening web. An ultrasound transducer is distally positioned relative to the distal end of the catheter shaft. The ultrasound transducer includes a piezoelectric transducer body. A balloon surrounds the ultrasound transducer. The first fluid lumen is configured to provide a cooling fluid to the balloon at a pressure and a flow rate sufficient to protect the ultrasound transducer and non-target tissue of the blood vessel from thermal injury. A single electrical cable extends through the cable lumen. The single electrical cable electrically connects to the ultrasound transducer. The single electrical cable is configured to deliver sufficient electrical energy during sonication to the transducer such that the transducer thermally induces modulation of neural fibers surrounding the blood vessel sufficient to improve a measurable physiological parameter corresponding to a diagnosed condition of the patient.

In accordance with certain embodiments, a tissue treatment system comprises a controller including a signal generator. The tissue treatment system includes a treatment catheter including a catheter shaft having an outer wall sized and shaped for delivery through a radial artery to a blood vessel of a patient. The catheter shaft has several lumens extending longitudinally through the catheter shaft between a distal end and a proximal end. The several lumens include a guidewire lumen within a guidewire lumen wall, and a cable lumen. The catheter shaft includes a stiffening web extending from the guidewire lumen wall. The several lumens include a first fluid lumen defined between the outer wall, the guidewire lumen wall, and the stiffening web. An outer wall thickness of the outer wall is less than a stiffening wall thickness of the stiffening web. An ultrasound transducer is distally positioned relative to the distal end of the catheter shaft. The ultrasound transducer includes a piezoelectric transducer body. A balloon surrounds the ultrasound transducer. The first fluid lumen is configured to provide a cooling fluid to the balloon at a pressure and a flow rate sufficient to protect the ultrasound transducer and non-target tissue of the blood vessel from thermal injury. A single electrical cable extends through the cable lumen. The single electrical cable electrically connects the signal generator to the ultrasound transducer. The single electrical cable is configured to deliver sufficient electrical energy during sonication to the transducer such that the transducer thermally induces modulation of neural fibers surrounding the blood vessel sufficient to improve a measurable physiological parameter corresponding to a diagnosed condition of the patient.

In accordance with certain embodiments, a method comprises advancing a treatment catheter through a radial artery to a blood vessel of a patient. The treatment catheter includes a catheter shaft having an outer wall, and several lumens extending longitudinally through the catheter shaft between a distal end and a proximal end. The several lumens include a guidewire lumen within a guidewire lumen wall, and a cable lumen. The catheter shaft includes a stiffening web extending from the guidewire lumen wall. The several lumens include a first fluid lumen defined between the outer wall, the guidewire lumen wall, and the stiffening web. An outer wall thickness of the outer wall is less than a stiffening wall thickness of the stiffening web. An ultrasound transducer is distally positioned relative to the distal end of the catheter shaft. The ultrasound transducer includes a piezoelectric transducer body. A balloon surrounds the ultrasound transducer. The first fluid lumen is configured to provide a cooling fluid to the balloon at a pressure and a flow rate sufficient to protect the ultrasound transducer and non-target tissue of the blood vessel from thermal injury. A single electrical cable extends through the cable lumen. The single electrical cable electrically connects to the ultrasound transducer. The single electrical cable is configured to deliver sufficient electrical energy during sonication to the transducer such that the transducer thermally induces modulation of neural fibers surrounding the blood vessel sufficient to improve a measurable physiological parameter corresponding to a diagnosed condition of the patient. The method comprises applying a voltage to the ultrasound transducer, using the single electrical cable, to cause the piezoelectric transducer body to generate ultrasonic waves.

This summary is not intended to be a complete description of the embodiments of the present technology. Other features and advantages of the embodiments of the present technology will appear from the following description in which the preferred embodiments have been set forth in detail, in conjunction with the accompanying drawings and claims.

### BRIEF DESCRIPTION OF THE DRAWINGS

The various features of the present disclosure and the manner of attaining them will be described in greater detail with reference to the following description, claims, and drawings, wherein reference numerals are reused, where appropriate, to indicate a correspondence between the referenced items, and wherein:

FIG. 1 illustrates selected components of an ultrasound-based tissue treatment system in accordance with certain embodiments of the present technology.

FIG. 2A illustrates a side view of selected components of the ultrasound-based tissue treatment system introduced in FIG. 1.

FIG. 2B illustrates a perspective view of additional selected components of the ultrasound-based tissue treatment system inserted into a body lumen in according to various configurations provided herein.

FIG. 2C illustrates a longitudinal cross-sectional view of a distal portion of a catheter of the ultrasound-based tissue treatment system in accordance with an embodiment of the present technology.

FIG. 2D illustrates how a balloon that surrounds an ultrasound transducer and is in apposition with a body lumen can be used to center the transducer within the body lumen.

FIG. 2E illustrates how one or more flexible baskets and/or extensions that expand distal and/or proximal of an ultrasound transducer may be used to center the transducer within a body lumen.

FIG. 2F illustrates how a basket that surrounds a transducer and a balloon may be used to center the transducer within a body lumen.

FIG. 3A1 illustrates a cross-sectional view of a catheter shaft, along the line A-A in FIG. 2C, in accordance with an embodiment.

FIG. 3A2 illustrates a cross-sectional view of the catheter shaft, along the line A-A in FIG. 2C, in accordance with an alternative embodiment.

FIG. 3B illustrates a cross-sectional view across a portion of the ultrasound transducer of the catheter, along the line B-B in FIG. 2C.

FIG. 3C illustrates example details of the cartridge and reservoir introduced in FIG. 1.

FIG. 4A illustrates a side view of a distal portion of the catheter according to an embodiment.

FIG. 4B illustrates a perspective view of the distal portion of the catheter shown in FIG. 4A.

FIG. 4C illustrates a cross-sectional view of two separate coaxial cables that can be used to provide the electrical cabling of the catheter shown in FIG. 2A, in accordance with an embodiment.

FIG. 5 illustrates a cross-sectional view of a shielded pair cable that can be used to provide the electrical cabling of the catheter shown in FIG. 2A, in accordance with an embodiment.

FIG. 6 illustrates a cross-sectional view of an alternative shielded pair cable that can be used to provide the electrical cabling of the catheter shown in FIG. 2A, in accordance with an embodiment.

FIG. 7 illustrates a cross-sectional view of two separate cables having solid wire inner conductors that can be used to provide the electrical cabling of the catheter shown in FIG. 2A, in accordance with an embodiment.

FIG. 8 illustrates a cross-sectional view of a single cable that includes a pair of solid wire inner conductors that can be used to provide the electrical cabling of the catheter shown in FIG. 2A, in accordance with an embodiment.

FIG. 9 illustrates a cross-sectional view of two separate cables having stranded wire inner conductors that can be used to provide the electrical cabling of the catheter shown in FIG. 2A, in accordance with an embodiment.

FIG. 10 illustrates a cross-sectional view of a single cable that includes a pair of stranded wire inner conductors that can be used to provide the electrical cabling of the catheter shown in FIG. 2A, in accordance with an embodiment.

FIG. 11A1 illustrates a cross-sectional view of a catheter shaft, along the line A-A in FIG. 2C, in accordance with an embodiment of the present technology.

FIG. 11A2 illustrates a cross-sectional view of the catheter shaft, along the line A-A in FIG. 2C, in accordance with another embodiment of the present technology.

FIG. 11A3 illustrates a cross-sectional view of a catheter shaft, along the line A-A in FIG. 2C, in accordance with a further embodiment of the present technology.

FIG. 11A4 illustrates a cross-sectional view of the catheter shaft, along the line A-A in FIG. 2C, in accordance with still another embodiment of the present technology.

FIG. 11A5 illustrates a cross-sectional view of the catheter shaft, along the line A-A in FIG. 2C, in accordance with still another embodiment of the present technology.

FIG. 12 is a high-level flow diagram that is used to summarize methods according to various embodiments of the present technology.

FIG. 13A illustrates a cross-sectional view of a single cable that includes a pair of solid wire inner conductors and a stranded wire conductor that can be used to provide the electrical cabling of the catheter shown in FIG. 2A, in accordance with an embodiment.

FIG. 13B illustrates a cross-sectional view of a single cable that includes a pair of solid wire inner conductors and a stranded wire conductor that can be used to provide the electrical cabling of the catheter shown in FIG. 2A, in accordance with an embodiment.

FIG. 14 illustrates a cross-sectional view of a catheter shaft, along the line A-A in FIG. 2C, in accordance with another embodiment of the present technology.

FIG. 15A illustrates a cross-sectional view of a single cable that includes a pair of solid wire inner conductors and a stranded wire conductor that can be used to provide the electrical cabling of the catheter shown in FIG. 2A, in accordance with an embodiment.

FIG. 15B illustrates a cross-sectional view of a single cable that includes a pair of solid wire inner conductors and a stranded wire conductor that can be used to provide the electrical cabling of the catheter shown in FIG. 2A, in accordance with an embodiment.

FIG. 16 illustrates a cross-sectional view of a catheter shaft, along the line A-A in FIG. 2C, in accordance with another embodiment of the present technology.

FIG. 17 illustrates a cross-sectional view of a catheter shaft, along the line A-A in FIG. 2C, in accordance with another embodiment of the present technology, wherein electrical cabling is integrated into the catheter shaft itself.

FIG. 18 illustrates a cross-sectional view of a catheter shaft, along the line A-A in FIG. 2C, in accordance with still another embodiment of the present technology, wherein electrical cabling is integrated into the catheter shaft itself.

FIG. 19 illustrates a cross-sectional view of a catheter shaft, along the line A-A in FIG. 2C, in accordance with still another embodiment of the present technology, wherein electrical cabling is integrated into the catheter shaft itself.

FIG. 20 illustrates example details of the controller introduced in FIG. 1.

FIG. 21 illustrates a cross-sectional view of a catheter shaft, along the line A-A in FIG. 2C, in accordance with another embodiment of the present technology.

FIG. 22 illustrates a cross-sectional view of a catheter shaft, along the line A-A in FIG. 2C, in accordance with another embodiment of the present technology.

### DETAILED DESCRIPTION

Acoustic-based tissue treatment transducers, apparatuses, systems, and portions thereof, are provided herein. Preferably, the systems are catheter-based and may be delivered intraluminally (e.g., intravascularly) so as to place a transducer within a target anatomical region of the subject, for example, within a suitable body lumen such as a blood vessel. Once properly positioned within the target anatomical region, the transducer can be activated to deliver unfocused ultrasonic energy radially outwardly so as to suitably heat, and thus treat, tissue within the target anatomical region. The transducer can be activated at a frequency, duration, and energy level suitable for treating the targeted tissue. The transducer may be surrounded by a balloon configured to be at least partially filled with a cooling fluid that is supplied to the balloon via at least one lumen of the catheter. The at least one lumen of the catheter carrying the cooling fluid may have a cross-sectional area suitable to support a flow rate of the cooling fluid that cools the transducer while protecting the wall of the lumen from injury and supporting proper priming of the system such as to remove air bubbles that could have a negative impact on energy transmission. In one nonlimiting example, the unfocused ultrasonic energy generated by the transducer may target select nerve tissue of the subject, and may heat such tissue in such a manner as to neuromodulate (e.g., fully or partially ablate, necrose, or stimulate) the nerve tissue. In a manner such as described in the Warnking, Schaer, and Taylor patents mentioned above, neuromodulating renal nerves may have a positive impact on the progression various conditions, e.g., hypertension, chronic kidney disease, atrial fibrillation, heart failure, end stage renal disease, myocardial infarction, anxiety, contrast nephropathy, diabetes, metabolic disorder and insulin resistance, fatty liver disease, autoimmune disorders, sleep apnea, sleep disorders, cancer, depression, eating disorders, Loin Pain Hematuria Syndrome (LPHS), post-traumatic stress disorder, contrast nephropathy, arrhythmia, atrial fibrillation, polycystic kidney disease, autosomal dominant polycystic kidney disease (ADPKD), pain related to polycystic kidney disease, acute myocardial infarction, or cardio-renal syndrome. For example, ultrasound renal denervation has been shown to significantly decrease blood pressure and have a therapeutic benefit to patients diagnosed with hypertension in several clinical trials.

However, it should be appreciated that the transducers suitably may be used to treat other nerves and conditions, e.g., sympathetic nerves of the hepatic plexus within a hepatic artery responsible for blood glucose levels important to treating diabetes, or any suitable tissue, e.g., heart tissue triggering an abnormal heart rhythm, e.g., nerves of the celiac trunk to denervate the liver, spleen, and/or pancreas, e.g., afferent nerves of the pancreas causing pancreatic pain, e.g., denervation of the splenic artery and splenic nerves and/or superior mesenteric artery and plexus and/or inferior mesenteric artery and plexus to treat inflammatory disorders, e.g., colitis, inflammatory bowel disease, etc., denervation of the pulmonary artery to treat pulmonary hypertension, and is not limited to use in treating (e.g., neuromodulating) renal nerve tissue.

Embodiments of ultrasound denervation catheter systems disclosed herein may be used to improve one or more measurable physiological parameter corresponding to a diagnosed condition of the patient, such as blood pressure, insulin resistance, diabetic control, renin levels, renal catecholamine (e.g., norepinephrine) spillover, metanephrine, urinary sodium excretion, urinary potassium excretion, urine catecholamines, pain, AF, congestion, or other neurohormones, lipid profile, liver enzyme, blood sugar, liver function, hemoglobin A1C, etc.

In certain embodiments, a catheter suitable for radial access through the radial artery of the arm is provided. Radial access is a more comfortable route of delivery than through the groin and is associated with fewer complications. In order to provide a catheter suitable for tissue treatment via a radial access point, it is advantageous to provide a longer length catheter than is required for access through the femoral artery. However, because of the increase in length, the cross-sectional area of fluid lumens must be increased in order to maintain a pressure and fluid flow rate suitable for safe and effective energy delivery and for priming of the system, which may be required to remove air bubbles. However, because a radial device will be threaded through the blood vessels of the arm, which tend to be smaller than the blood vessels associated with femoral artery delivery, it is advantageous for the overall diameter of the catheter to be decreased to 5F or smaller for patient comfort and safety. Certain embodiments provide a catheter having a cross-sectional area and length suitable for radial access, while including one or more fluid lumens having a cross-sectional area suitable for providing a safe and effective nerve ablation and priming of the system.

In certain embodiments, the catheter includes a cable system having a decrease in cross-sectional area, while being suitable to carry the large amount of power necessary to ablate nerves to create a therapeutic effect. In certain embodiments, the catheter may include a cable lumen that is smaller in cross-sectional area to advantageously permit one or more fluid lumens of the catheter to be larger in cross-sectional area, while permitting the overall diameter of the radial access catheter (or a smaller femoral access catheter) to shrink in relation to a 6 F catheter, e.g., a 6 F femoral access catheter. As used herein, the diameter of the catheter means the diameter of the largest part of the catheter, e.g., the larger of the transducer or the catheter shaft diameter.

In certain embodiments, a catheter is provided that includes a cable system within one or more fluid lumens and omits a separate cable lumen. In certain embodiments, the catheter includes a cable system within the lumen shaft and omits a separate cable lumen, thereby advantageously permit one or more fluid lumens of the catheter to be larger in cross-sectional area, while permitting the overall diameter of the radial access catheter (or a smaller femoral access catheter) to shrink in relation to a 6 F catheter. In certain embodiments, a 5 F catheter is provided that includes a water-backed transducer having a frequency of about 9 MHz. In certain embodiments, a 4 F catheter is provided that includes a water-backed transducer having a frequency of about 12-16 MHz. In certain embodiments, a balloonless catheter is provided, which may omit one or more fluid lumens. In certain embodiments, a balloonless catheter is provided, which includes a fluid lumen configured to transmit cooling fluid to the transducer, but does not include a return fluid lumen.

### Overview of System Components and Features

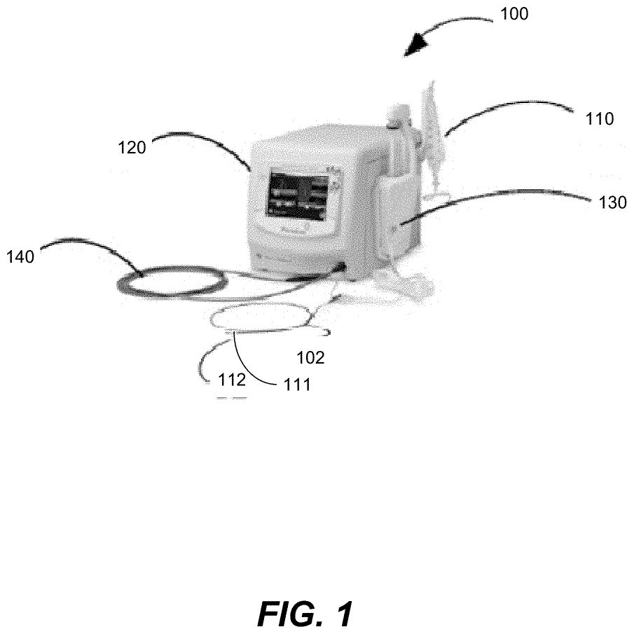

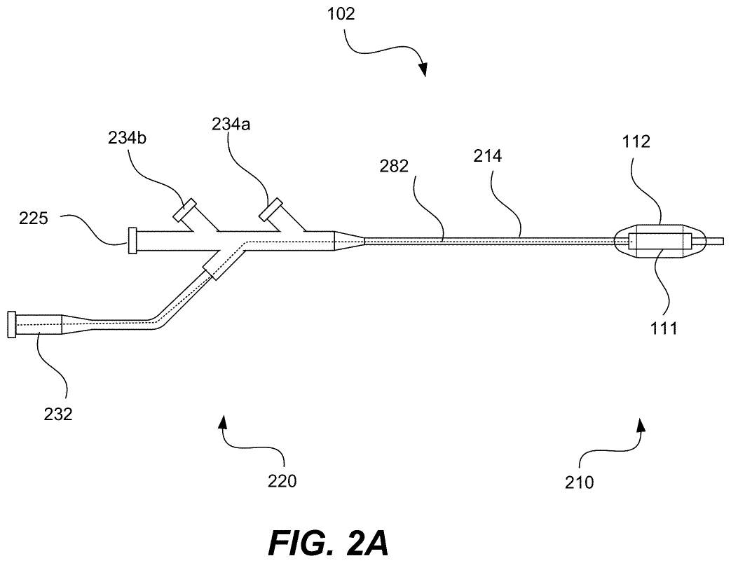

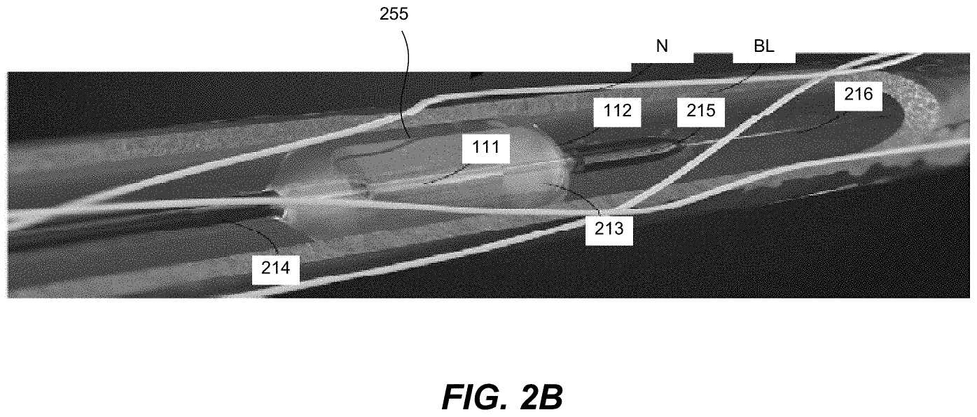

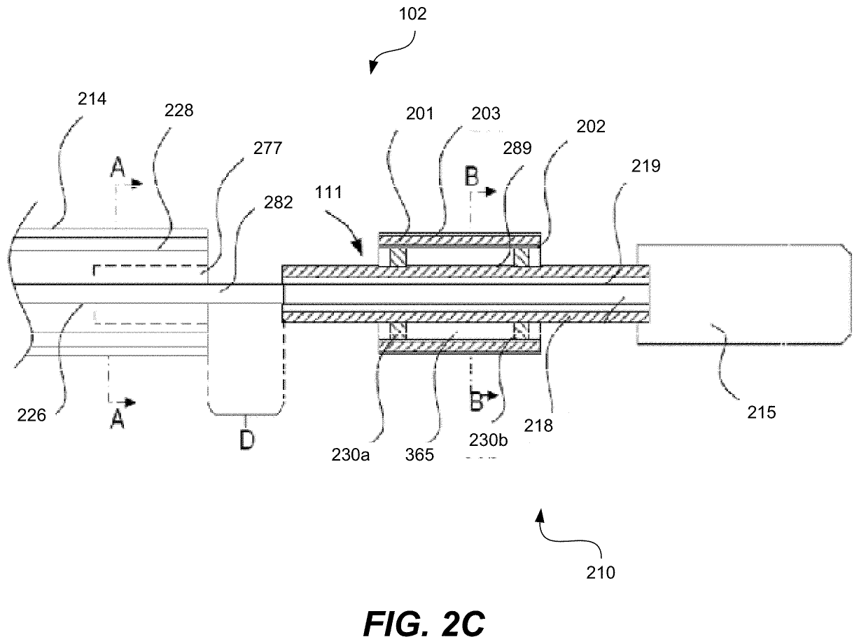

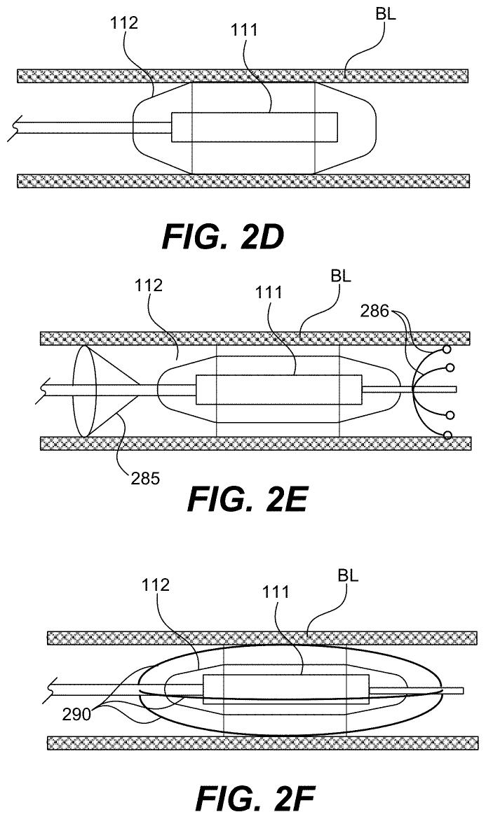

FIGS. 1, 2A, and 2B illustrate features of an ultrasound-based tissue treatment system 100, according to various configurations provided herein. Referring initially to FIG. 1, the system 100 is shown as including a catheter 102, a controller 120, and a connection cable 140. In certain embodiments, the system 100 further includes an ultrasound transducer 111 within a balloon 112, a reservoir 110, a cartridge 130, and a control mechanism, such as a handheld remote control. In certain embodiments, which can be referred to as “balloonless” embodiments, the system 100 does not include the balloon 112. In certain such balloonless embodiments, the system 100 also does not include the reservoir 110 and the cartridge 130. In certain other balloonless embodiments, the system 100 does include the reservoir 110 and/or the cartridge 130.

In the embodiment shown in FIG. 1, the controller 120 is shown as being connected to the catheter 102 through the cartridge 130 and the connection cable 140. In certain embodiments, the controller 120 interfaces with the cartridge 130 to provide a cooling fluid to the catheter 102 for selectively inflating and deflating the balloon 112. The balloon 112 can be made, e.g., from nylon, a polyimide film, a thermoplastic elastomer (such as those marked under the trademark PEBAX™), a medical-grade thermoplastic polyurethane elastomers (such as those marketed under the trademark PELLETHANE™), pellethane, isothane, or other suitable polymers or any combination thereof, but is not limited thereto.

Referring now to FIG. 2A, the catheter 102 includes a distal portion 210 and a proximal portion 220. The catheter 102 includes a catheter shaft 214, which can include one or more lumens extending therethrough. For an example, the catheter shaft 214 includes a guidewire lumen 225 that is shaped, sized and otherwise configured to receive a guidewire. In certain embodiments suitable, e.g., for renal denervation, the catheter 102 is 6 French and the catheter shaft 214 has an on outer diameter of about 0.07 inches. In certain embodiments suitable, e.g., for renal denervation, the catheter 102 is 5 French and the catheter shaft 214 has an on outer diameter of about 0.055 inches and a length of at least 145 cm. In certain embodiments suitable, e.g., for renal denervation, the catheter 102 is 4 French and the catheter shaft 214 has an on outer diameter of about 0.04 inches and a length of at least 145 cm.

In certain embodiments suitable, e.g., for renal denervation, the catheter 102 is 5 French and the catheter shaft 214 has an on outer diameter of about 0.055 inches and a length of at least 150 cm. In certain embodiments suitable, e.g., for renal denervation, the catheter 102 is 4 French and the catheter shaft 214 has an on outer diameter of about 0.04 inches and a length of at least 145 cm.

In an embodiment suitable for femoral access, the catheter 102 has a 6 French diameter, the catheter shaft 214 has an on outer diameter of about 0.07 inches, and the length of the catheter shaft 214 is about 85 cm, and the cross-sectional area of each of the fluid lumens 327 and 328 is about 0.0004 cm² to 0.0006 cm², but is not limited thereto.

In an embodiment suitable for femoral or radial access, the catheter 102 has a 5 French diameter, the catheter shaft 214 has an outer diameter of about 0.055 inches, the length of the catheter shaft 214 is about 145 cm to 175 cm, the cross-sectional area of each of the fluid lumens is about 0.0006 cm² to 0.0008 cm², but is not limited thereto.

In an embodiment suitable for femoral or radial access, the catheter 102 has a 4 French diameter, the catheter shaft 214 has an outer diameter of about 0.04 inches, the length of the catheter shaft 214 is about 145 cm to about 175 cm, the cross-sectional area of each of the fluid lumens is about 0.0006 cm² to 0.0008 cm², but is not limited thereto.

The proximal portion 220 of the catheter 102 may include one or more connectors or couplings. For example, the proximal portion 220 may include one or more electrical coupling(s) 232. The catheter 102 may be coupled to the controller 120 by connecting the electrical coupling(s) 232 to the connection cable 140. The connection cable 140 may be removably connected to the controller 120 and/or the catheter 102 via a port on the controller 120 and/or the catheter 102, in order to permit use of multiple catheters during a procedure. In certain embodiments, e.g., where only one catheter 102 needs to be used during a procedure, the connection cable 140 may be permanently connected to the controller 120. As will be described in additional detail below, electrical cabling (represented by the dashed line 282 in FIG. 2A) that extends through at least one lumen of the catheter 102 electrically couples the transducer 111 to the electrical coupling(s) 232. The controller 120 includes a signal generator that applies a voltage between the electrodes of the transducer 111 to thereby cause the transducer to generate ultrasonic waves. In accordance with certain embodiments, a characteristic impedance of the electrical cabling 282 is matched to a characteristic impedance of the transducer 111 and to a characteristic output impedance of the signal generator (of the controller 120) to which the electrical cabling 282 is configured to be coupled.

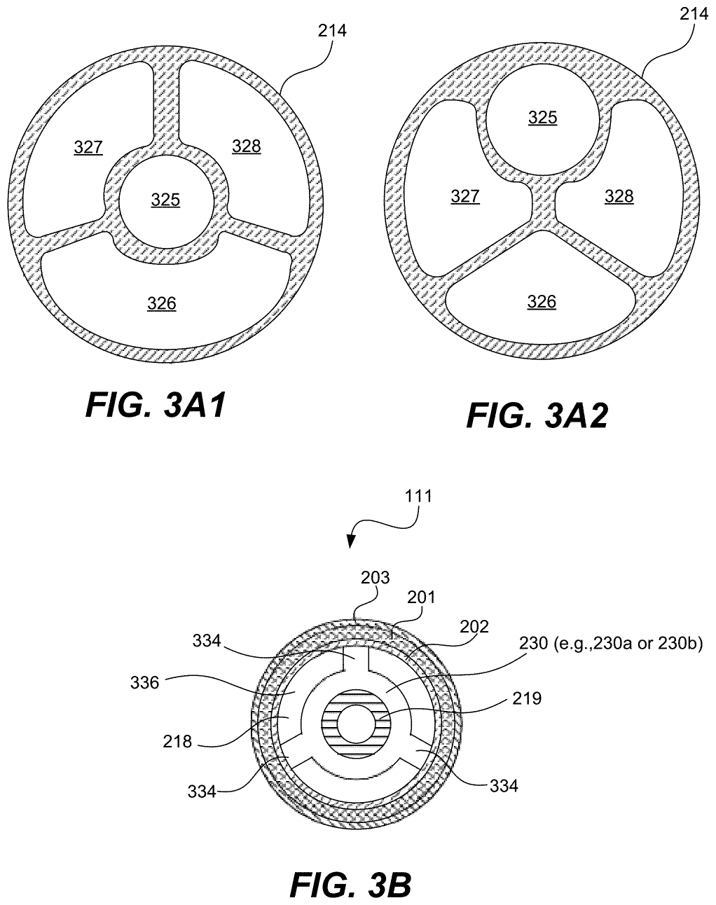

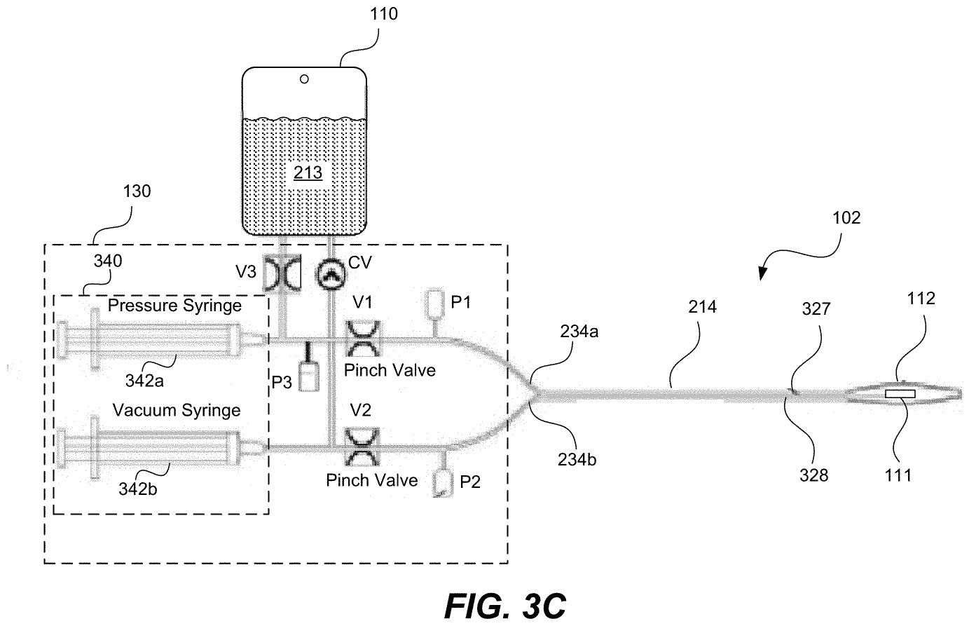

In certain embodiments, the proximal portion 220 of the catheter 102 may further include one or more fluidic ports, e.g., a fluidic inlet port 234a and a fluidic outlet port 234b, via which an expandable member (e.g., balloon 112) may be fluidly coupled to the cartridge 130 (shown in FIG. 1), which supplies cooling fluid. The cartridge 130 optionally may be included within controller 120, attached to the outer housing of controller 120 as shown in FIG. 1, or may be provided separately. In other embodiments, the fluidic inlet port 234a and the fluidic outlet port 234b, the balloon 112, and the cartridge 130 may all be absent from the system 100. Other variations are also possible and within the scope of the embodiments described herein. Example details of the cartridge 130 and the reservoir 110 are described below with reference to FIG. 3C.

FIG. 2B illustrates a perspective view of selected components of the catheter 102, e.g., components of the distal portion 210 as may be inserted into a body lumen BL of a subject. In FIG. 2B, the body lumen BL is a blood vessel (e.g., a renal artery) that has a plurality of nerves N in an outer layer (e.g., adventitia layer) of the blood vessel. As illustrated in FIG. 2B, the distal portion 210 may include the ultrasound transducer 111, the balloon 112 filled with a cooling fluid 213, the catheter shaft 214, and/or a guidewire support tip 215 configured to receive a guidewire 216.

The transducer 111 may be disposed partially or completely within the balloon 112, which may be inflated with a cooling fluid 213 so as to contact the interior surface (e.g., intima) of the body lumen BL. In certain embodiments, the transducer 111 may be used to output an acoustic signal when the balloon 112 fully occludes a body lumen BL. The balloon 112 may center the transducer 111 within the body lumen BL. In certain embodiments, e.g., suitable for renal denervation, the balloon 112 is inflated while inserted in the body lumen BL of the patient during a procedure at a working pressure of about 1.4 to 2 atm using the cooling fluid 213. The balloon 112 may be or include a compliant, semi-compliant or non-compliant medical balloon. The balloon 112 is sized for insertion in the body lumen BL and, in the case of insertion into the renal artery, for example, the balloon 112 may be selected from available sizes including outer diameters of 3.5, 4.2, 5, 6, 7, or 8 mm, but not limited thereto. In some embodiments, as shown in FIG. 2B, when inflated by being filled with the cooling fluid 213 under the control of the controller 120, the outer wall of the balloon 112 may be generally parallel with the outer surface of the transducer 111. Optionally, the balloon 112 may be inflated sufficiently as to be in apposition with the body lumen BL. For example, when inflated, the balloon 112 may at least partially contact, and thus be in apposition with, the inner wall of the body lumen BL.

In other configurations, the balloon 112 is configured not to contact the body lumen BL when expanded. The balloon 112 may surround the transducer in order to cool the transducer during sonications, but the balloon may not contact or occlude the body lumen BL, and the blood within the body lumen BL may be relied upon to cool the body lumen BL instead of the cooling fluid. In addition, instead of relying on the balloon 112 to center the transducer, in certain embodiments, one or more flexible baskets that expand proximal and distal of the transducer 111 may be used to center the transducer 111. In an alternative embodiment, at least one balloon that expands proximal and distal of the transducer 111 may be used to center the transducer 111. In another embodiment, a basket that surround the transducer 111 and balloon 112 may be used to center the transducer, which basket is preferably made of a material that does not interfere with the sonication.

In certain embodiments, wherein the balloon 112 surrounds the transducer, but the balloon does not contact or occlude the body lumen BL, the balloon 112 is non-compliant. In certain embodiments, the balloon 112 comprises nylon. The catheter may use a single size non-compliant balloon/sheath coupled with centering mechanisms located proximal and distal to the balloon to center the device within the vessel such that native flow of the blood is not obstructed. The cooling of the artery wall is provided by the native blood flow while cooling and the isolation of the transducer from the blood stream is provided within the balloon. In an embodiment, the non-compliant balloon of a fixed diameter that is less than or equal to the minimum arterial size to be treated, e.g., a 3.5 mm balloon may be used for an artery that is at least 3.5 mm in diameter. For vessels greater than the non-compliant balloon size, centering mechanisms will be provided such that the ultrasound treatment zone and blood flow within the vessel is unobstructed. This mechanism prevents arterial overstretching at the treatment site during ablation. The cooling will be provided to the arterial wall by the native blood flow that exists within the vessel during treatment.

For a blood vessel that matches the balloon diameter, the non-complaint balloon (e.g., 112) will act as the centering mechanism. The cooling of the arterial wall will be managed by the generator's cooling system by flowing water through the balloon if necessary. A non-compliant balloon advantageously offers tighter control of balloon design. The non-compliant balloon (e.g., 112) may be advantageously constructed such that the balloon surface does not interfere with sonication and holds the desired shape.

Additionally, or alternatively, the balloon 112 may be maintained at a specified size by pushing cooling fluid into and pulling cooling fluid out of the balloon 112 at a specified flow rate. In balloonless embodiments, the transducer 111 is not disposed within a balloon.

In certain balloonless embodiments, the fluid lumens are configured to maintain a flow rate sufficient to cool the transducer and/or protect non-target tissue of the blood vessel.

In certain embodiments having a balloon around the transducer but not occluding the blood vessel, the fluid lumens are configured to maintain a flow rate sufficient to cool the transducer and/or protect non-target tissue of the blood vessel.

FIG. 2C illustrates a longitudinal cross-sectional view of the distal portion 210 of the catheter 102. FIG. 3A1 illustrates a cross-sectional view of the catheter shaft 214 along the line A-A shown in FIG. 2C, according to an embodiment. FIG. 3A2 illustrates a cross-sectional view of the catheter shaft 214 along the line A-A shown in FIG. 2C, according to an alternative embodiment. FIG. 3B illustrates a cross-sectional view of the ultrasound transducer 111 along the line B-B shown in FIG. 2C, according to an embodiment. In certain embodiments, the catheter shaft 214 may be about 1.8 mm in diameter. The catheter shaft 214 can be made, e.g., from a thermoplastic elastomer (such as those marked under the trademark PEBAX™), a medical-grade thermoplastic polyurethane elastomers (such as those marketed under the trademark PELLETHANE™), pellethane, isothane, or other suitable polymers or any combination thereof, but is not limited thereto. The catheter shaft 214 includes one or more lumens that may be used as fluid conduits, an electrical cabling passageway, a guidewire lumen and/or the like, as described in further detail below with reference to FIGS. 3A1 and 3A2.

In certain embodiments suitable, e.g., for renal denervation, via femoral access, the guidewire 216 has a diameter of about 0.36 mm and a length of from about 180 cm to about 300 cm, and is delivered using a 7 French guide catheter, having a minimum inner diameter of 2.06 mm and a length less than about 80 cm. In certain embodiments, a 6 French guide catheter is used to deliver the guidewire 216. In certain embodiments, the guide catheter has a length of about 55 cm. In certain embodiments, the guide catheter has a length of about 85 cm and a hemostatic valve is attached to the hub of the guide catheter to allow for continuous irrigation of the guide catheter to decrease the risk of thromboembolism.

A 6 French or 5 French guide catheter may be advantageously used to deliver a 5 French or 4 French tissue treatment catheter. The length of the guide catheter may be extended for radial access, e.g., to 145 cm or greater.

Referring again to FIG. 2C, the ultrasound transducer 111 may include a cylindrical hollow tube 201 made of a piezoelectric material (e.g., lead zirconate titanate (PZT), etc.), with inner and outer electrodes 202, 203 disposed on the inner and outer surfaces of the cylindrical tube 201, respectively. Such a cylindrical hollow tube of piezoelectric material is an example of, and thus can be referred to as, a piezoelectric transducer body 201. The piezoelectric transducer body 201 can have various other shapes and need not be hollow. In certain embodiments suitable, e.g., for renal denervation, the piezoelectric material, of which the piezoelectric transducer body 201 is made, is lead zirconate titanate 8 (PZT8), which is also known as Navy III Piezo Material. Raw PZT transducers may be plated with layers of copper, nickel and/or gold to create electrodes on surfaces (e.g., the inner and outer surfaces) of the piezoelectric transducer body (e.g., 201), as disclosed in U.S. Pat. No. 10,230,041 to Taylor, incorporated herein by reference in its entirety. Application of a voltage and alternating current across inner and outer electrodes 202, 203 causes the piezoelectric material to vibrate transverse to the longitudinal direction of the cylindrical tube 201 and radially emit ultrasonic waves. While the ultrasound transducer 111 in FIG. 2C is not shown as being surrounded by a balloon, it is noted that the ultrasound transducer 111 can be positioned within a balloon (e.g., 112), e.g., as shown in FIGS. 2A and 2B.

As shown in FIG. 2C, the ultrasound transducer 111 is generally supported via a backing member or post 218. In certain embodiments, the backing member 218 (which can also be referred to as the post 218) comprises stainless steel coated with nickel and gold, wherein nickel is used as a bonding material between the stainless steel and gold plating. In certain embodiments suitable, e.g., for renal denervation, an outer diameter of the transducer 111 is about 1.5 mm, an inner diameter of the transducer 111 is about 1 mm, and the transducer 111 has a length of about 6 mm. Transducers having other inner diameters, outer diameters, and lengths, and more generally dimensions and shapes, are also within the scope of the embodiments described herein. Further, it is noted that the drawings in the FIGS. are not necessarily drawn to scale, and often are not drawn to scale.

As illustrated in FIG. 2C, the backing member 218 may extend from the distal portion 210 of the catheter shaft 214 to a distal tip 215. For example, the distal end of the backing member 218 may be positioned within an adjacent opening in the tip 215, and the proximal end of the backing member 218 may be moveably coupled to the distal portion 210 of the catheter shaft 214 via the electrical cabling 282. In other embodiments, there is a gap (e.g., labeled D in FIG. 2C) between the distal end of the catheter shaft 214 and the proximal end of the ultrasound transducer 111. Additional details of the electrical cabling 282, and how the electrical cabling may be electrically coupled to the transducer 111, are described below.

In order to permit liquid cooling along both the inner and outer electrodes 202, 203, the backing member 218 may include one or more stand-off assemblies 230a and 230b. The stand-off assemblies 230a, 230b may define one or more annular openings through which cooling fluid 213 may enter the space of the transducer 111 (which may be selectively insulated) between the backing member 218 and the inner electrode 202. Accordingly, the backing member 218 may serve as a fluid barrier between the cooling fluid 213 circulated within the balloon 112 and the lumen of the backing member 218 that receives the guidewire 216. As shown schematically in FIG. 2C, for example, the stand-off assemblies 230a, 230b of the backing member 218 may be positioned along or adjacent to each longitudinal end of the ultrasound transducer 111 (separated by a main post body 289) and couple the cylindrical tube 201 of the ultrasound transducer 111 to the backing member 218. With reference to FIG. 3B, a stand-off assembly 230 (230a or 230b) may have a plurality of lugs, ribs, or attachment points 334 that engage the inner electrode 202 of the transducer 111. In certain embodiments, the attachment points 334 are soldered to the inner electrode 202 of the transducer 111. The number, dimensions, and placement of the ribs 334 may vary, as desired or required. For example, as illustrated in FIG. 3B, a total of three ribs 334 can be generally equally-spaced apart from one another at an angle of 120 degrees apart from one another, defining openings 336 through which a cooling fluid or blood may enter an interior space of the cylindrical tube 201 between the inner electrode 202 disposed along the inner surface of the cylindrical tube 201 and the backing member 218. In certain embodiments, the maximum outer diameter of stand-off assemblies 230a and 230b is about 1 mm, the outer diameter of the main post body 289 is about 0.76 mm, and the inner diameter of backing member 218 is about 0.56 mm.

In accordance with certain embodiments, the stand-off assemblies 230a, 230b are electrically conductive, so as to electrically couple the inner electrode 202 of the ultrasound transducer 111 to the backing member 218. One or more conductors of the electrical cabling 282 may be electrically coupled to the backing member 218. Thus, as the controller 120 is activated (or more specifically, the signal generator thereof is activated), current may be delivered from the electrical cabling 282 to the inner electrode 202 of the ultrasound transducer 111 via the backing member 218 and the stand-off assemblies 230a, 230b, which advantageously eliminates the need to couple the cabling 282 directly to the inner electrode 202 of the transducer 111. In other embodiments, the backing member 218 and the stand-off assemblies 230a, 230b are made of one or more electrical insulator material(s), or if made of an electrically conductive material(s) are coated with one or more electrical insulator material(s). In certain embodiments, one or more electrical conductors of the cabling 282 are directly coupled (e.g., soldered) to the inner electrode 202 of the transducer 111.

Moreover, as illustrated in FIG. 2C, the backing member 218 may have an isolation tube 219 disposed along its interior surface so as to prevent or reduce the likelihood of electrical conduction between the guidewire 216 (shown in FIG. 2B) and the backing member 218, for use in embodiments where such an electrical conduction is not desired. The isolation tube 219 can be formed of a non-electrically conductive material (e.g., a polymer, such as polyimide), which can also be referred to as an electrical insulator. As illustrated in FIG. 2C, the isolation tube 219 may extend from the catheter shaft 214 through the lumen of the backing member 218 within the transducer 111 to the tip 215. In this manner, the transducer 111 is distally offset from the distal end of the catheter shaft 214.

As illustrated in FIG. 2C, the catheter 102 may also include a bore 277 extending from the distal end of the catheter 102 proximally within the catheter 102, and sized and shaped to receive at least a portion of the backing member 218, thereby electrically insulating the isolation tube 219 and/or the ultrasound transducer 111. Accordingly, during delivery of the catheter 102 to the anatomical region being treated, the backing member 218, the isolation tube 219, and/or the ultrasound transducer 111 may be at least partially retracted within the bore 277 of the catheter 102, e.g., by retracting the electrical cabling 282, thereby providing sufficient stiffness to the catheter 102 such that the catheter 102 may be delivered in a safe manner.

FIG. 2B shows a distal portion 210 of the catheter 102 inserted into a body lumen (BL), such as a renal artery, such that the balloon 112 when sufficiently inflated with cooling fluid is in apposition with the body lumen BL. As noted above, in alternative embodiments, the balloon 112 may surround the transducer 111 in order to cool the transducer during sonications, but the balloon 112 may not contact or occlude the body lumen BL, and the blood within the body lumen BL may be relied upon to cool the body lumen BL instead of the cooling fluid. Referring to FIG. 2E, in certain such embodiments, instead of relying on the balloon 112 to center the transducer 111, one or more flexible baskets 285 and/or extensions 286 that expand distal and/or proximal of the transducer 111 may be used to center the transducer 111. In an alternative embodiment, at least one balloon that expands proximal and/or distal of the transducer 111 may be used to center the transducer 111. Referring to FIG. 2F, in another embodiment, a basket 290 that surrounds the transducer 111 and the balloon 112 may be used to center the transducer 111, which basket 290 is preferably made of a material that does not interfere with the sonications.

As illustrated in FIGS. 3A1 and 3A2, the catheter shaft 214 includes one or more lumens that can be used as fluid conduits, electrical cabling passageways, guidewire lumen, and/or the like. For example, as illustrated in FIGS. 3A1 and 3A2, the catheter shaft 214 may comprise a guidewire lumen 325 that is shaped, sized and otherwise configured to receive the guidewire 216. In certain embodiments, as illustrated in FIG. 3A1, the guidewire lumen 325 is located in the center of the catheter shaft 214 in order to center the transducer 111 within the catheter shaft 214. Alternatively, the guidewire lumen 325 can be offset from the center of the catheter shaft 214, e.g., as shown in FIG. 3A2. The catheter shaft 214 may also include a cable lumen 326 for receiving electrical cabling 282. Further, the catheter shaft 214 can include one or more fluid lumens 327, 328 for transferring the cooling fluid 213 (e.g., water, sterile water, saline, 5% dextrose (D5 W)), other liquids or gases, etc., from and to a fluid source, e.g., the reservoir 110 and cartridge 130, at the proximal portion 220 of the catheter 102 (external to the patient) to the balloon 112 under control of the controller 120. Active cooling of about the first millimeter of tissue is designed to preserve the integrity of the blood vessel wall, e.g., the renal arterial wall. The guidewire lumen 325 can extend longitudinally through the entire catheter shaft 214, parallel to the fluid lumens 327, 328. Alternatively, the guidewire lumen 325 may extend longitudinally through only a portion of the catheter shaft 214, e.g., where the catheter 102 is a rapid exchange (Rx) type of catheter.

The catheter 102 may include only a single fluid lumen or two or more fluid lumens (e.g., 3, 4, more than 4, etc.), as desired or required. As illustrated in FIG. 3A1, in an embodiment, the fluid lumens 327 and 328 and the cable lumen 326 all have a kidney-shaped or D-shaped cross-sections configured to maximizes efficiency of fluid flow delivery and distribute fluid uniformly across the ultrasound transducer 111 by maximizing area, while minimizing the perimeter of the fluid lumens 327 and 328. In certain embodiments, each of the fluid lumens 327 and 328 and the cable lumen 326 are substantially symmetrical, the same size, the same geometry, and/or are interchangeable, e.g., as shown in FIG. 3A1. Changes in fluid flow rate within the catheter can lead to delayed, incomplete, or over treatment. In certain embodiments, the catheter shaft 214 is configured to enable a fluid flow rate of about 40 mL/min. In certain embodiments, the catheter shaft 214 is configured to enable a fluid flow rate of about 35 to 45 mL/min. In certain embodiments, the catheter shaft 214 is configured to enable a fluid flow rate of about 20 to 45 mL/min. In certain embodiments, e.g., suitable for radial delivery during a renal denervation procedure, the catheter shaft 214 is configured to enable a fluid flow rate of about 10 to 20 mL/min. Each of one or more lumens (e.g., 328) may be in fluid communication with the same or separate, individual fluid sources external to the patient at the proximal portion 220 of the catheter 102.

As another example, the catheter shaft 214 may include any suitable number of fluid lumens for transferring the cooling fluid to and from the balloon 112 (or to the transducer 111 in balloonless embodiments) from the reservoir 110 and cartridge 130 responsive to instructions executed by the controller 120. In certain balloonless embodiments, the catheter shaft 214 may omit fluid lumens 327, 328 and the system 100 may omit the reservoir 110 and the cartridge 130. In certain balloonless embodiments, the catheter shaft 214 includes the fluid lumens 327, 328 and the system 100 includes the reservoir 110 and the cartridge 130.

In certain embodiments, as illustrated in FIG. 3A2, the guidewire lumen 325 is located proximal to and/or shares a wall with the catheter shaft 214 so as to enable expedited exchange of catheters during a procedure. In such embodiments, the cable lumen 326 may be located opposite the guidewire lumen 225 and also share a wall with the catheter shaft 214. The cable lumen 326 may be, e.g., triangular or rectangular in shape, and may be configured to maximize the area available for and minimize the perimeter of the fluid lumens 327 and 328, thereby enabling a higher flow rate for the same pressure. The fluid lumens 327 and 328 may be shaped so as to optimize flow rate and reduce, and preferably minimize, fluidic friction. In such embodiments, the area of fluid lumens 327 and 328 may not be maximized, but instead the walls of the fluid lumens 327 and 328 may be rounded to avoid pockets that may otherwise cause fluidic friction, thereby optimizing flow rate of the cooling fluid 213 within the fluid lumens 327 and 328.

The catheter shaft 214 may include within at least the cable lumen 326, the electrical cabling 282 (e.g., a coaxial cable, parallel coaxial cables, a shielded parallel pair cable, one or more wires, or one or more other electrical conductors) coupling the inner and outer electrodes 202, 203 of the ultrasound transducer 111 to the controller 120, such that the controller 120 may apply a suitable voltage across such electrodes so as to cause the piezoelectric material of the transducer 111 to emit ultrasonic energy to a subject. In certain embodiments, the cable lumen 326 is shaped, sized and otherwise configured to receive the electrical cabling 282 (e.g., coaxial cable(s), wire(s), other electrical conductor(s), etc.). The electrical cabling 282 permits the electrodes 202, 203 of the ultrasound transducer 111 to be selectively activated in order to emit acoustic energy to a subject. More specifically, the electrical cabling 282 can allow for the communication of transducer information, such as operating frequency and power, from the catheter 102 to the controller 120 and/or vice versa, as well as the transfer of electrical energy to the ultrasound transducer 111 during a procedure.

The distal portion 210 of the catheter 102 may be percutaneously delivered to the target anatomical location (e.g., at a specified location within the body lumen BL) via any suitable intraluminal access route, e.g., via a gastrointestinal route or via an intravascular route such as the femoral or radial route. In certain embodiments, the controller 120 is configured so as to fill the balloon 112 with the cooling fluid 213 only after the distal portion 210 of the catheter 102 is suitably positioned at the target anatomical location. The catheter 102 may be delivered through the body lumen BL with or without the assistance of a commercially-available guidewire. For example, the catheter 102 and the balloon 112 may be delivered over the guidewire 216 (shown in FIG. 2B) and through a renal guide catheter. For further examples of guidewire-based delivery of ultrasound transducers, see U.S. Pat. No. 10,456,605, which was incorporated herein by reference above. However, it should be appreciated that any suitable steerable catheter or sheath, or any other suitable guiding device or method, may be used to deliver the distal portion 210 of the catheter 102 to a target anatomical location of the subject. Once delivered to a suitable location within the body lumen BL, the balloon 112 may be inflated with the cooling fluid 213 (e.g., under control of controller 120), and the transducer 111 may be actuated (e.g., by applying a voltage across the inner and outer electrodes 202, 203 under control of the controller 120) so as to deliver unfocused ultrasonic energy to the target anatomical location. The transducer 111 is sized for insertion in the body lumen BL and, in the case of insertion of the renal artery, for example, the transducer 111 may have an outer diameter of less than 2 mm, for example, about 1.5 mm and an inner diameter of less than 1.8 mm, for example, about 1 mm. As described in greater detail below, the length L of the transducer 111 optionally may be selected such that the ultrasonic waves that it generates has a near field depth suitable for generating a lesion only within a desired region relative to the wall of a target body lumen BL.

It will be appreciated that the frequency, power, and amount of time for which the transducer 111 is actuated suitably may be selected based on the treatment to be performed. The period of time during which the transducer 111 is actuated may be sufficient to complete the particular treatment being performed, and may depend on factors such as the power at the transducer, the frequency of ultrasonic energy emitted, the size of the tissue region being treated, the age, weight and gender of the patient being treated, and/or the like. Illustratively, in some configurations the time period for which the transducer 111 may be actuated may be in a range of about 7 seconds to 15 seconds. In some configurations the time period for which the transducer 111 may be actuated may be in a range of about 7 seconds to 12 seconds. In some configurations the time period for which the transducer 111 may be actuated may be in a range of about 7 seconds to 10 seconds. Or, for example, the transducer 111 may be actuated for less than 13 seconds (s), e.g., 7 seconds. Decreasing the duration of sonication advantageously decreases the amount of pain related to the procedure.

In various configurations, the delivery of ultrasound energy during the treatment may be continuous or substantially continuous, e.g., without any interruptions or fluctuations in frequency, power, duty cycle and/or any other parameters. Alternatively, one or more of the frequency, power, duty cycle, or any other parameter may be modified during the treatment. For example, in some configurations, the delivery of ultrasonic energy is modulated, e.g., between on and off, or between a relatively high level and a relatively low level, so as prevent or reduce the likelihood of overheating of adjacent (e.g., targeted or non-targeted) tissue. For examples of such modulation, see U.S. Pat. No. 10,499,937 to Warnking, the entire contents of which are incorporated herein by reference.

In example configurations in which nerve tissue is to be treated, e.g., the nerves N illustrated in FIG. 2B, the transducer 111 may be positioned and configured so as to deliver ultrasonic energy through the wall of a body lumen BL that is adjacent to that nerve tissue, e.g., through the wall of the body lumen BL. In one nonlimiting example, renal nerves to be treated using the transducer 111 may be located about 0.5 mm to 8 mm (e.g., about 1 mm to 6 mm) from the inner wall of the renal artery. In other examples, nerve tissue to be treated may be located less about 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, less than 0.5 mm, or more than 8 mm from the inner wall of a body lumen in which transducer is disposed. Under control of the controller 120, the transducer 111 generates unfocused ultrasonic energy that heats any suitable nerve tissue so as to at least partially neuromodulate such nerve tissue, e.g., cause complete or partial ablation, necrosis, or stimulation of such nerve tissue. The ultrasonic energy generated by the transducer 111 may radiate radially outward so as to target the nerve tissue regardless of the radial orientation of such nerve tissue relative to the body lumen. In some configurations, the unfocused ultrasonic energy is delivered along an entire, continuous circumference of the transducer 111. In other configurations, the ultrasonic energy is emitted non-continuously or intermittently around the circumference of the transducer 111. It should be appreciated that nerve tissue, and more specifically the renal nerves, are only one example of tissue that may be treated using an ultrasound transducer. Other examples of target anatomical regions that may be treated with an ultrasound transducer 111 are described elsewhere herein.

Additional options regarding designs and uses of ultrasound transducers and catheter-based ultrasound delivery systems are provided in the following patents and published applications, the entire contents of each of which are incorporated by reference herein: U.S. Pat. Nos. 6,635,054; 6,763,722; 7,540,846; 7,837,676; 9,707,034; 9,981,108; 10,350,440; 10,456,605; 10,499,937; and PCT Publication No. WO 2012/112165.

In accordance with certain embodiments of the present technology, the piezoelectric transducer body of the ultrasound transducer comprises a hollow tube of piezoelectric material having an inner surface and an outer surface. In certain such embodiments, a first electrode is disposed on one of the inner and outer surfaces of the hollow tube of piezoelectric material, and a second electrode is disposed on the other one of the inner and outer surfaces of the hollow tube of piezoelectric material. The hollow tube of piezoelectric material can be cylindrically shaped, such that it has a circular shaped radial cross-section. In certain embodiments suitable, e.g., for renal denervation, the piezoelectric material, of which the piezoelectric transducer body is made, is lead zirconate titanate 8 (PZT8), which is also known as Navy III Piezo Material. Raw PZT transducers may be plated with layers of copper, nickel and/or gold to create electrodes on surfaces (e.g., the inner and outer surfaces) of the piezoelectric transducer body. In alternative embodiments the hollow tube of piezoelectric material can have other shapes besides being cylindrical with a circular cross-section. Other cross-sectional shapes for the hollow tube of piezoelectric material, and more generally the piezoelectric transducer body, include, but are not limited to, an oval or elliptical cross-section, a square or rectangular cross-section, pentagonal cross-section, a hexagonal cross-section, a heptagonal cross-section, an octagonal cross-section, and/or the like. In still other embodiments, the piezoelectric transducer body is not hollow, e.g., can have a generally solid rectangular shape, or some other solid shape.

In certain embodiments, the piezoelectric transducer body can be configured, e.g., to deliver acoustic energy in a frequency range of 8.5 to 9.5 MHz, but is not limited thereto. In certain embodiments, wherein the catheter is a 5F or larger catheter, the piezoelectric transducer body can be configured, e.g., to deliver acoustic energy in a frequency range of 8.5 to 9.5 MHz, but is not limited thereto. In accordance with certain embodiments, the piezoelectric transducer body is configured to produce an acoustic output power within a range of 20 to 45 Watts in response to an input electrical power within a range of 25 to 50 Watts, but is not limited thereto. In accordance with certain embodiments, the controller 120 is configured to output a power of about 50 W into 63 ohms at 7 seconds on, having an output frequency of about 8.5-9.5 MHz. In accordance with certain embodiments, the controller 120 is configured to output a power of about 25 W to 50 W at 7 to 10 seconds on, having an output frequency of about 8.5-9.5 MHz. In accordance with certain embodiments, the controller 120 is configured to output 300 J to 350 J into the catheter. In accordance with certain embodiments, the catheter is configured to deliver about 200 J to 250 J into the balloon. In accordance with certain embodiments, the cable is configured to deliver at least 25 W to the transducer, such as to provide sufficient power to denervate nerve surrounding a blood vessel. In accordance with certain embodiments, the cable is configured to deliver 25 W to 40 W to the transducer, such as to provide sufficient power to denervate nerve surrounding a blood vessel. In accordance with certain embodiments, the cable and the transducer are impedance matched.

In accordance with certain embodiments, the controller 120 is configured to output a power of about 15 W to 35 W at 7 to 12 seconds on, having an output frequency of about 12-16 MHz. In accordance with certain embodiments, the catheter is a 4F catheter and the controller 120 is configured to output a power of about 15 W to 35 W at 7 to 12 seconds on, having an output frequency of about 12-16 MHz.

In certain embodiments, the piezoelectric transducer body is configured to produce an acoustic output power within a range of 25 to 50 Watts in response to an input electrical power within a range of 10 to 80 Watts, but is not limited thereto.

Example details of the cartridge 130 and the reservoir 110, which were introduced above in the above discussion of FIG. 1, will now be described with reference to FIG. 3C. Referring to FIG. 3C, the reservoir 110 is shown as being implemented as a fluid bag, which can be the same or similar to an intravenous (IV) bag in that it can hang from a hook, or the like. The reservoir 110 and the cartridge 130 can be disposable and replaceable items.

The reservoir 110 is fluidically coupled to the cartridge 130 via a pair of fluidic paths, one of which is used as a fluid outlet path (that provides fluid from the reservoir to the cartridge), and the other one of which is used as a fluid inlet path (the returns fluid from the cartridge to the reservoir). The cartridge 130 is shown as including a syringe pump 340, which includes a pressure syringe 342a and a vacuum syringe 342b. Each of the pressure syringe 342a and the vacuum syringe 342b includes a respective barrel, plunger, and hub, with the hub of each of the syringes 342a, 342b coupled to a respective fluid tube or hose. The cartridge 130 is also shown as including pinch valves V1, V2 and V3, pressure sensors P1, P2, and P3, and a check valve CV. While not specifically shown in FIG. 3C, the syringe pump 340 can include one or more gears and step-motors, and/or the like, which are controlled by the controller 120 (in FIG. 1) to selectively maneuver the plungers of the pressure syringe 342a and the vacuum syringe 342b. Alternatively, the gear(s) and/or step-motor(s) can be implemented within the controller 120, and can be used to control the syringe pump 340.

In order to at least partially fill the barrel of the pressure syringe 342a with a portion of the cooling fluid that is stored in the reservoir 110, the check valve CV and the pinch valves V1 and V2 are closed, the pinch valve V3 is opened, and the plunger of the pressure syringe 342a is pulled upon to draw cooling fluid into the barrel of the of the pressure syringe 342a. The pinch valve V3 is then closed and the pinch valve V1 is opened, and then the plunger of the pressure syringe 342a is pushed upon to expel cooling fluid from the barrel of the pressure syringe 342a through the fluid tube attached to the hub of the pressure syringe 342a. The cooling fluid expelled from the pressure syringe 342a enters the fluid lumen 327 (in the catheter shaft 214), via the fluidic inlet port 234a of the catheter 102, and then enters and at least partially fills the balloon 112. Depending on various factors, such as the volume of the barrel of the pressure syringe 342a and the volume of the balloon 112, and/or the like, the process just described above may need to be performed multiple times in order to fill the balloon 112 with cooling fluid to a desired fluid pressure, e.g., of 10 pounds per square inch (psi), but not limited thereto.

In order to remove the cooling fluid from the balloon 112, the pinch valve V1 is closed and the pinch valve V2 is opened, and then the plunger of the vacuum syringe 342b if pulled on to draw the cooling fluid in the balloon through the fluid lumen 328, through the fluidic outlet port 234b, and through the fluid tube connected to the hub of the vacuum syringe 342b, thereby drawing the cooling fluid into the barrel of the vacuum syringe 342b.

In order to return the cooling fluid from the barrel of the vacuum syringe 342b to the reservoir 110, the pinch valves V1, V2, and V3 are all closed, the check valve CV is opened, and the plunger of the vacuum syringe 342b is pushed on to expel the cooling fluid out of the barrel of the vacuum syringe 342b, past the check valve CV, and into the reservoir 110.

The pressure sensors P1, P2, and P3 can be used to monitor the fluidic pressure at various points along the various fluidic paths within the cartridge 130, which pressure measurements can be provided to the controller 120 as feedback that is used for controlling the syringe pump 340 and/or for other purposes, such as, but not limited to, determining the fluidic pressure within the balloon 112.

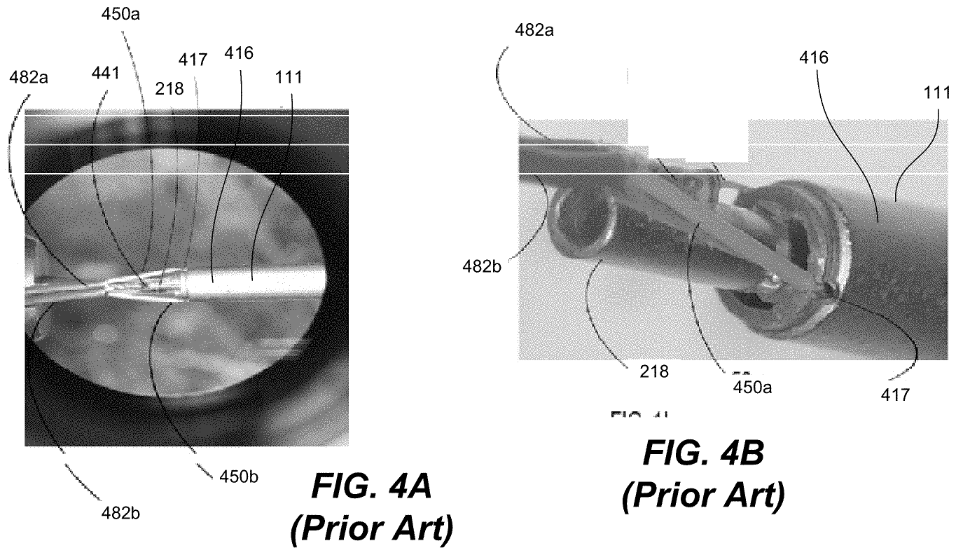

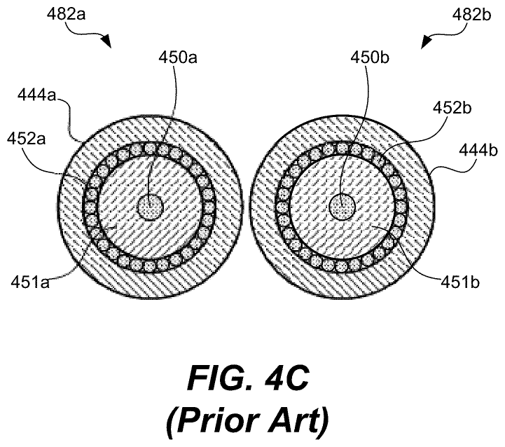

FIGS. 4A and 4B illustrate, respectively, side and perspective views of a distal portion of the catheter 102 according to an embodiment. As can be seen in FIGS. 4A and 4B, in accordance with certain embodiments, the ultrasound transducer 111 includes a non-stepped portion 416 and a stepped portion 417. In certain embodiments, the axial length of stepped portion 417 is about 0.4 mm and the axial length of a non-stepped portion 416 is about 6 mm. In certain embodiments, the non-stepped portion 416 of the transducer 111 is the portion of the transducer that produces the bulk of the ultrasonic energy delivered from the catheter 101. In certain embodiments, the stepped portion 417 has a different outer-diameter than the non-stepped portion 416 and behaves in a different manner than the non-stepped portion 416. In certain embodiments, a cylindrical portion of the balloon 112 (shown, e.g., in FIG. 2B) is at least as long as the length of non-stepped portion 416 in order to provide cooling protection equivalent to the length of the transducer 111. In certain embodiments, wherein the axial length of a non-stepped portion 416 is about 6 mm, the cylindrical portion 255 of balloon 112 has a length at least about 6 mm.

In intraluminal systems, ultrasound transducers may be disposed within balloons that are filled with a cooling fluid before and during treatment. Alternatively, an ultrasound transducer may be exposed directly to the bloodstream, without a surrounding balloon, in what may be referred to as balloonless embodiments. In certain balloonless embodiments, blood in the body lumen, e.g., renal artery, is relied upon to cool both the transducer and the artery lumen. In certain balloonless embodiments, the transducer may still be cooled using cooling fluid. For example, cooling fluid may flow through and/or around the body of the ultrasonic transducer without using a balloon, for example by using a hollow region on the outside and/or inside of the transducer for fluid to flow. In certain embodiments, a balloon may surround the transducer, and the balloon may contact the interior surface (e.g., intima) of the body lumen BL. In certain embodiments, the transducer may be used to output an acoustic signal when the balloon fully occludes a body lumen BL, and the cooling fluid within the balloon may be used to cool both the body lumen and the transducer. In certain embodiments, the balloon may surround the transducer in order to cool the transducer during sonications, but the balloon may not contact or occlude the body lumen, and the blood within the body lumen may be relied upon to cool the body lumen instead of the cooling fluid.

### Electrical Cabling Embodiments

Parallel Pair of Coaxial Cables

FIGS. 4A and 4B, which were briefly discussed above, also shown two separate coaxial cables 482a, 482b electrically connected to a proximal portion of the ultrasound transducer 111. The two separate coaxial cables 482a, 482b can collectively comprise the electrical cabling 282 introduced above in the discussion of FIG. 2C. FIG. 4C illustrates a cross-sectional view of the two separate coaxial cables 482a, 482b. Referring to FIG. 4C, the coaxial cable 482a includes an inner conductor 450a surrounded by a dielectric 451a, which is surrounded by an outer tubular conducting shield 452a, all of which are surrounded by an insulating jacket 444a. Similarly, the coaxial cable 482b includes an inner conductor 450b surrounded by a dielectric 451b, which is surrounded by an outer tubular conducting shield 452b, all of which are surrounded by an insulating jacket 444b. In certain embodiments, the inner conductors 450a, 450b each comprise one wire constructed, e.g., of 42 AWG (American Wire Gauge) solid silver-plated copper alloy. The dielectrics 451a, 451b can each comprise a polymer, e.g., perfluoro alkoxy (PFA), having an outer diameter of about 0.007 inches, but are not limited thereto. The outer diameter of each of the coaxial cables 482a, 482b can be about 0.0125 inches, but are not limited thereto. The outer tubular conducting shields 452a and 452b can each be constructed, e.g., of a plurality of 48 AWG silver-plated copper wires. In certain embodiments suitable, e.g., for renal denervation, the coaxial cables 482a, 482b may each have a characteristic impedance of about 50 ohms and, when used in parallel, can have a combined characteristic impedance of about 25 ohms.

To maximize the efficiency of the system 100, it is desirable minimize reflections at the various interfaces between the electrical cabling 282 and the controller 120 (via the connection cable 140) and between the electrical cabling 282 and the transducer 111. This can be achieved by having the characteristic impedances of the connector at the controller 120, the connection cable 140, the electrical cabling 282, and the transducer 111, substantially match one another. Nevertheless, embodiments of the present technology also cover systems where the aforementioned characteristic impedances do not substantially match one another.

In an embodiment suitable, e.g., for renal denervation, the combined cross-sectional areas of the coaxial cables 482a and 482b is about 0.00031 square inches and the width may be about 0.025 inches, when the coaxial cables 482a and 482b are positioned adjacent to and touching one another. A reason that the combined width of the pair of coaxial cables 482a and 482b is so small, and that it would not be practical to use a pair of coaxial cables having a substantially larger combined width, is that the pair of coaxial cables 482a and 482b need to fit within and be fed through a lumen (e.g., cable lumen 326) of the catheter shaft 214. Examples of the cable lumen 326 were shown in and described above with reference to FIGS. 3A1 and 3A2.

Referring back to FIGS. 4A and 4B, the distal ends of the inner conductors 450a, 450b of the coaxial cables 482a, 482b can be electrically coupled to the outer electrode 203 on the proximal end of the transducer 111 approximately 180 degrees apart. More specifically, as illustrated in FIGS. 4A and 4B, each of the inner conductors 450a, 450b of the coaxial cables 482a, 48b may be soldered on the stepped portion 417 on the proximal end of the transducer 111 about 180 degrees apart, such that current output from the controller 120 flows from the inner conductors 450a, 450b of the coaxial cables 282a, 282b to the outer electrode 203 of the transducer 111. The proximal ends of the inner conductors 450a, 450b of the coaxial cables 482a, 482b (which proximal ends are not shown in the FIGS.) can be electrically coupled to one another by soldering, or the like. When located within the cable lumen 326 of the catheter shaft 214, the pair of coaxial cables 482a, 482b will extend through the cable lumen 326 parallel to one another.

Each of the outer conductors 452a, 452b may comprise multiple wires, as can be appreciated from the cross-sectional view shown in FIG. 4C. The wires of outer tubular conducting shields 452a, 452b can all be bundled together (e.g., see bundle 441 in FIG. 4B) and soldered to the backing member 218, so as to electrically couple the inner electrode 202 of the transducer 411 to the coaxial cables 482a, 482b via the backing member 218 (in embodiments where the backing member is electrically conductive and electrically coupled to the inner electrode 202). Current may then travel back to controller 120 by flowing from the controller 120 to the inner electrode 202 to the backing member 218 to the outer tubular conducting shields 452a, 452b of the coaxial cables 482a, 482b. The proximal ends of the outer tubular conducting shields 452a, 452b of the coaxial cables 482a, 482b (which proximal ends are not shown in the FIGS.) can be similarly bundled together and/or soldered together.

In other embodiments, the conductors of coaxial cables 482a, 482b can be reversed. In other words, the distal ends of the inner conductors 450a, 450b of the coaxial cables 482a, 482b can be electrically connected to the inner electrode 202 (e.g., by being soldered to the backing member 218), the wires of outer tubular conducting shields 452a can all be bundled together and electrically coupled to the outer electrode 203 by soldering to the stepped portion 417, and the wires of the outer tubular conducting shields 452b can all be bundled together and electrically coupled to the outer electrode 203 by soldering to the stepped portion 417 about 180 degrees from where the bundled together wires of outer tubular conducting shields 452a are soldered. In still other embodiments, if the inner conductors 450a, 450b are large enough in diameter to handle the amount of current needed to power the ultrasound transducer 111, then one of the inner conductors 450a, 450b can be electrically coupled to the inner electrode 202, the other one the inner conductors 450a, 450b can be electrically coupled to the outer electrode 203, and the outer tubular conducting shields 452a and 452b can be commonly grounded.

Using the two separate coaxial cables 482a, 482b in the manner described above with reference to FIGS. 4A-4C advantageously increases the efficiency of the system and decreases the amount of heat generated during an unfocused ultrasound ablation procedure by decreasing the amount of electrical energy lost to heat, compared to using a single coaxial cable (e.g., 482a), where the distal end of the inner conductor (e.g., 450a) of the single coaxial cable is electrically coupled to the outer electrode 203 and the wires of outer tubular conducting shield (e.g., 452a) of the single coaxial cable are bundled together and soldered to the backing member 218 to thereby electrically couple the outer tubular conducting shield to inner electrode 202.

Shielded Parallel Pair Cables

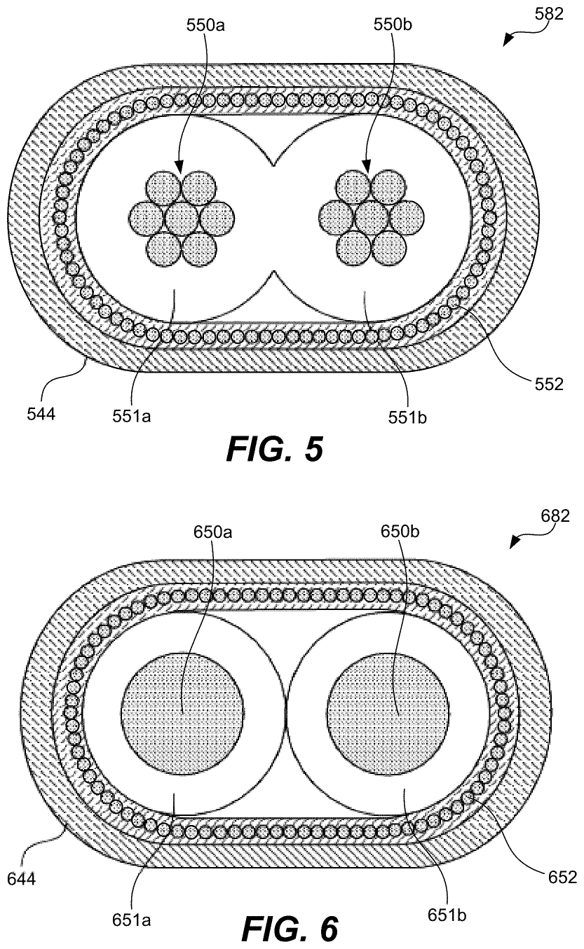

FIG. 5 shows a cross-sectional view of another embodiment of the electrical cabling 282 of the catheter 102. According to this embodiment, the electrical cabling 282 comprises a shielded parallel pair cable 582 comprising parallel inner conductors 550a and 550b, each surrounded by a respective dielectric 551a and 551b, with the dielectric pair surrounded by an outer tubular conducting shield 552, which is surrounded by an insulation jacket 544. In one embodiment, the shielded parallel pair cable 582 has a height of about 0.0125 inches and a width of about 0.020 inches. Each of the inner conductors 550a and 550b may be constructed, e.g., of 42 AWG stranded silver-plated copper alloy wire, which comprise multiple (e.g., seven) wires. The dielectrics 551a, 551b may comprise a polymer, e.g., perfluoroalkoxy (PFA), having an outer diameter of about 0.007 inches. The outer tubular conducting shield 552 may be constructed, e.g., of multiple wires of 48 AWG silver-plated copper wire. In an embodiment suitable, e.g., for renal denervation, the shielded parallel pair cable 582 has a cross-sectional area of about 0.00025 square inches, which represents about a 20 percent reduction in cross-sectional area compared to the combined cross-sectional area of coaxial cables 482a and 482b. Accordingly, the parallel pair cable 582 takes up less room within a specific lumen (e.g., one of the cable lumens 326) of a catheter shaft (e.g., 214) than the pair of coaxial cables 482a and 482b collectively, or can fit into a lumen having a smaller cross-sectional area than is needed to fit the pair of coaxial cables 582a and 582b. It is noted that the terms “electrical cabling,” “cabling,” and “cable” are often used interchangeably herein.

In an embodiment, the wires of the inner conductor 550a may be coupled, e.g., twisted, together, and the wires of the inner conductor 550b may likewise be twisted together. Twisting the wires in the manner advantageous decreases the noise ratio, making the cable more efficient, i.e., leading to less loss of energy to heat. This reduction of heat generation may advantageously improve the integrity and lifespan of the transducer 111. The inner conductors 550a and 550b of the shielded parallel pair cable 582 can then be electrically coupled with the outer electrode 203 of the transducer 111 on the proximal end of the transducer 111, preferably approximately 180 degrees apart from one another, similar to as shown in FIGS. 4A and 4B. In an embodiment, the inner conductor 550a and the inner conductor 550b may be soldered on the stepped portion 417 about 180 degrees apart from one another, such that current output from the controller 120 flows from the inner conductors 550a, 550b of the shielded parallel pair cable 582 to the outer electrodes 203 of the transducer 111. The plurality of wires of the outer tubular conducting shield 552 can be bundled together and soldered to the backing member 218, so as to electrically couple the inner electrode 202 of the transducer 111 to the shielded parallel pair cable 582 via the backing member 218. Current may then travel back to the controller 120 by flowing from the inner electrode 202 to the backing member 218 to the outer tubular conducting shield 552 of the shielded parallel pair cable 582. In other embodiments, the conductors of the shielded parallel pair cable 582 can be reversed. That is, the inner conductors 550a and 550b can be bundled or twisted together and electrically coupled to the inner electrode 202 of the transducer 111, and the plurality of wires of the outer tubular conducting shield 552 can be bundled together and electrically coupled to the outer electrode 203 of the transducer (e.g., by being soldered to the stepped portion 417 of the outer electrode 203).

Using the shielded parallel pair cable 582 instead of two separate coaxial cables 582a, 582b (as was the case in FIG. 4C) significantly decreases the area taken up in catheter 102 by the cabling 582, advantageously enabling the catheter 102 to have a smaller diameter, while maintaining an adequate flow of the cooling fluid 213 through the fluid lumens (e.g., 327 and/or 328) in order to provide sufficient cooling of a body lumen BL during an ablation procedure and maintaining or even improving the current carrying capacity of the cabling 582. In addition, without prejudice or limitation, it is theorized that the multi-wire configuration of the inner conductors 550a and 550b further increases the efficiency of the system and decreases the amount of heat generated during an unfocused ultrasound ablation procedure by decreasing the amount of electrical energy lost to heat. This reduction of heat generation may advantageously improve the integrity and lifespan of the transducer 111.

In certain embodiments, the cable is a twisted parallel pair. In certain embodiments, the cable is a shielded twisted parallel pair. Twisting the cable advantageously makes the cable less vulnerable to electronic interferences and also makes the cable easily terminated. Because of the reduction of the electronic interference, the power output is more consistent, providing a safer more efficacious system for the patient. The reduction of the electronic interference also prevents malfunction of the electrical system due to sudden surges in power, increasing the life of the transducer and protecting transducer performance.

Similarly, shielding the parallel pairs provides for more control of electrical properties of the cable by ensuring less fluctuations in power output of the system caused by electrical interference from nearby equipment.

The reduction in the cross-sectional area of the cable enables the catheter shaft 214 to be reduced in size, e.g., from having a 6 French diameter to having a 5 French diameter, or even a 4 French diameter, while permitting an increase in the cross-sectional area and length of the fluid lumens, providing for improved radial access to the renal arteries, wherein the smaller the diameter the better it is to provide for easier and less painful access to the renal arteries. While achieving this reduction cross-sectional area of the cabling 582, cable 582 is configured to deliver at least 40 W to the transducer 111, thereby supplying sufficient electrical power to the transducer to achieve denervation of nerve fibers sufficient to significantly improve a measurable physiological parameter corresponding to a diagnosed condition of the patient.

In certain embodiments, system 100 may comprise a 4F to 5F radial access ultrasound balloon catheter sized and shaped for delivery through the radial artery to a blood vessel in a vicinity of neural fibers, the catheter comprising a cable 582 configured to deliver sufficient electrical power to the transducer to thermally induce modulation of target neural fibers sufficient to significantly improve a measurable physiological parameter corresponding to a diagnosed condition of the patient while protecting non-target tissue in the blood vessel wall from thermal injury.

In certain embodiments, system 100 may comprise a 4F to 5F radial access ultrasound balloon catheter sized and shaped for delivery through the radial artery to a renal artery, the catheter comprising a cable 582 configured to deliver sufficient electrical power to the transducer to thermally induce modulation of target neural fibers surrounding the renal artery sufficient to significantly improve a patient's hypertension while protecting non-target tissue in the renal artery from thermal injury.

FIG. 6 shows a cross-sectional view of another embodiment of electrical cabling 682 of the catheter 102. According to this embodiment, the electrical cabling 682 comprises a shielded parallel pair cable 682 comprising parallel solid wire inner conductors 650a and 650b, each surrounded by a respective dielectric 651a and 651b, with the dielectric pair surrounded by an outer tubular conducting shield 652, which is surrounded by an insulation jacket 644. In accordance with an embodiment, the shielded parallel pair cable 682 has a height of about 0.010 inches and a width of about 0.017 inches. In an embodiment, each of the inner conductors 650a and 650b is a single wire, e.g., 38 AWG solid silver-plated copper alloy having a width of about 0.004 inches. The outer tubular conducting shield 652 may be constructed, e.g., of multiple 50 AWG silver-plated copper wires. In an embodiment suitable for, e.g., renal denervation, the shielded parallel pair cable 682 has a cross-sectional area of about 0.00017 square inches, which represents about a 46 percent reduction in cross-sectional area compared to the combined cross-sectional area of the coaxial cables 482a and 482b.

The inner conductors 650a and 650b of the shielded parallel pair cable 682 can be electrically coupled with outer electrode 203 on the proximal end of the transducer 111 approximately 180 degrees apart from one another. In an embodiment, the inner conductors 650a, 650b are soldered on the stepped portion 417 of the transducer 411 about 180 degrees apart from one another, such that current output from controller 120 flows from inner conductors 650a, 650b of shielded parallel pair cable 682 to the outer electrodes 203 of the transducer 111. The plurality of wires of outer tubular conducting shield 652 can be bundled together and soldered to backing member 218, so as to electrically couple inner electrode 202 of the transducer 111 to shielded parallel pair cable 682 via the backing member 218. Current may then travel back to controller 120 by flowing from the inner electrode 202 to the backing member 218 to the outer tubular conducting shield 652 of the shielded parallel pair cable 682. In other embodiments, the conductors of the shielded parallel pair cable 682 can be reversed. It is believed that increasing the diameter (decreasing the gauge) of the inner conductors 650a, 650b of the cabling 682 may permit more current to be carried by cabling 682, thereby permitting more power to be used by the catheter 102.

Unshielded Cables

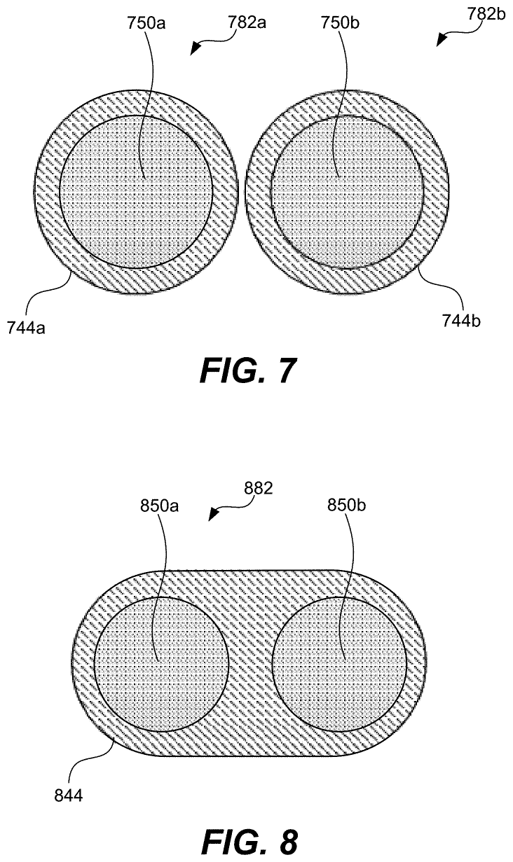

Referring now to FIG. 7, the electrical cabling 282 of the catheter 102 in this embodiment includes two separate cables 782a and 782b. The cable 782a includes a solid inner conductor wire 750a surrounded by an insulating jacket 744a. Similarly, the cable 782a includes a solid inner conductor wire 750b surrounded by an insulating jacket 744b. In an embodiment, the inner conductor wires 750a, 750b can each be, e.g., a 34 AWG solid silver-plated copper alloy wire. The inner conductor wire 750a of the cable 782a can be electrically coupled with outer electrode 203 on the proximal end of the transducer, e.g., by being soldered to the stepped portion 417 of the transducer 211. The inner conductor 750b of the cable 782b can be electrically coupled to the inner electrode 202 of the transducer, e.g., by being soldered to backing member 218. In an embodiment, the parallel cables 782a and 782b have a collective width of about 0.016 inches, a height of about 0.008 inches, and a cross-sectional area of about 0.00013 square inches, which represents about a 58 percent reduction in the combined cross-sectional area compared to the combined cross-sectional area of coaxial cables 482a and 482b.

Referring now to FIG. 8, the electrical cabling 282 of the catheter 102 in this embodiment is comprises the cabling 882 that includes a pair of solid inner conductor wires 850a and 850b surrounded by an insulating jacket 844. In an embodiment, the inner conductor wires 850a, 850b can each be, e.g., a 34 AWG solid silver-plated copper alloy wire. The inner conductor wire 850a of the cable 882 can be electrically coupled with outer electrode 203 on the proximal end of the transducer, e.g., by being soldered to the stepped portion 417 of the transducer 211. The inner conductor 850b of the cable 882 can be electrically coupled to the inner electrode 202 of the transducer, e.g., by being soldered to backing member 218. In an embodiment, the cable 282 has a width of about 0.020 inches, a height of about 0.010 inches, and a cross-sectional area of about 0.0002 square inches, which represents about a 36 percent reduction in the combined cross-sectional area compared to the combined cross-sectional area of coaxial cables 482a and 482b.

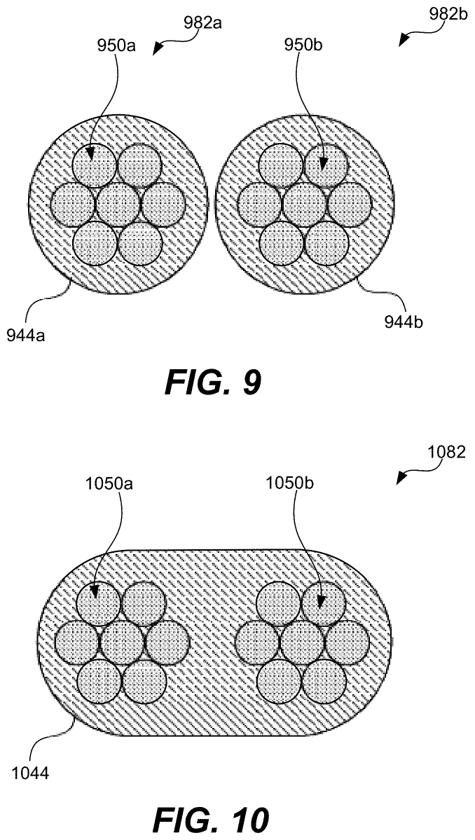

Referring now to FIG. 9, the electrical cabling 282 of the catheter 102 in this embodiment includes two separate cables 982a and 982b. The cable 982a includes an inner conductor 950a surrounded by an insulating jacket 944a. Similarly, the cable 982a includes an inner conductor 950b surrounded by an insulating jacket 944b. The inner conductors 950a, 950b can each comprise, e.g., a 34 AWG solid silver-plated copper alloy stranded wire that comprises a plurality of (e.g., seven) wires. In an embodiment, the parallel cables 982a and 982b have a collective width of about 0.020 inches, a height of about 0.010 inches, and cross-sectional area of about 0.0002 square inches, which represents about a 36 percent reduction in the combined cross-sectional area compared to the combined cross-sectional area of coaxial cables 482a and 482b. Without prejudice or limitation, it is theorized that because inner conductors 950a and 950b are each constructed from multiple wires, the efficiency of the system will increase, while heat generated during an unfocused ultrasound ablation procedure will be mitigated by decreasing the amount of electrical energy lost to heat compared systems utilizing cabling with single wire conductors. The wires may be twisted to shield the wires from electrical interference that could undesirably cause power surges in the system.

Referring now to FIG. 10, the electrical cabling 282 of the catheter 102 in this embodiment comprises the cabling 1082 that includes a pair of stranded wire inner conductors 1050a and 1050b surrounded by an insulating jacket 1044. In an embodiment, the inner conductor wires 850a, 850b can each be, e.g., a 34 AWG solid silver-plated copper alloy stranded wire that comprises a plurality of (e.g., seven) wires. The inner conductor wire 1050a of the cable 882 can be electrically coupled with outer electrode 203 on the proximal end of the transducer, e.g., by being soldered to the stepped portion 417 of the transducer 211. The inner conductor 1050b of the cable 882 can be electrically coupled to the inner electrode 202 of the transducer, e.g., by being soldered to backing member 218. In an embodiment, the cable 1082 has a width of about 0.020 inches, a height of about 0.010 inches, and a cross-sectional area of about 0.0002 square inches, which represents about a 36 percent reduction in the combined cross-sectional area compared to the combined cross-sectional area of coaxial cables 482a and 482b.

As can be appreciated from the above discussion of FIGS. 4A through 10, the electrical cabling 282 that provides power to the ultrasound transducer 111 can be implemented using a pair of distinct cables (e.g., 482a and 482b, 782a and 782b, or 982a and 982b), or using a single cable (e.g., 582, 682, 882, or 1082). The various different embodiments of the electrical cabling 282 (e.g., 582, 682, 782a and 782b, 882, 982a and 982b, and 1082) are especially useful for providing power to an ultrasound transducer (e.g., 111) of an intravascular therapeutic catheter, such as the catheter 102. More specifically, the electrical cabling 282 can be used to by the controller 120 to selectively provide a voltage between electrodes of a piezoelectric transducer body of the ultrasound transducer 111 to thereby cause the transducer 111 to generate ultrasonic waves. Referring briefly back to FIG. 2A, such electrical cabling 282 can extend between the proximal end of the transducer 111 and the electrical coupling 232. The electrical cabling can pass through a cable lumen (e.g., 326) of the catheter shaft 214. Alternatively, as will be described below, such electrical cabling, or at least a portion thereof, can pass through one or more of the fluid lumens of the catheter shaft 214, e.g., the fluid lumen(s) 327 and/or 328 in FIG. 3A1 or 3A2.

### Using Same Lumen(s) to Receive Cooling Fluid and Electrical Cabling

Referring briefly back to FIGS. 3A1 and 3A2, in the embodiments shown therein the catheter shaft 214 includes a total of four lumens, which include a guidewire lumen 325, a cable lumen 326, and a pair fluid lumens 327, 328. As explained above, the guidewire lumen 325 is for receiving a guidewire, the cable lumen 326 is for receiving the electrical cabling 282, and the fluid lumens 327, 328 are used for delivering cooling fluid (e.g., 213) to and from a balloon (e.g., 112) within which an ultrasound transducer (e.g., 111) is located. More specifically, the fluid lumens 327, 328 are used for transferring the cooling fluid 213 (e.g., water, sterile water, saline, 5% dextrose (D5 W)) from and to a fluid source, e.g., the reservoir 110 and the cartridge 130, at the proximal portion 220 of the catheter 102 (external to the patient) to the balloon 112 under control of the controller 120. In other words, the cooling fluid 213 can be circulated between the reservoir 110 and/or the cartridge 130 and the balloon 112 under the control of the controller 120. One or more fluid pumps, or the like, under the control of the controller 120 can be used to transfer the cooling fluid 213 between the reservoir 110 and/or the cartridge 130 and the balloon 112.

One of the fluid lumens (e.g., 327) is used to transfer (i.e., supply) the cooling fluid from the reservoir 110 and/or the cartridge 130 to the balloon 112, and the other one of the fluid lumens (e.g., 328) is used to transfer (i.e., return) the cooling fluid from the balloon 112 back to the reservoir 110 and/or the cartridge 130. In this manner, the cooling fluid can be used to provide active cooling of about the first millimeter of tissue to thereby preserve the integrity of the blood vessel wall, e.g., the renal arterial wall. The cooling fluid can also be used to cool the transducer 111, so that the temperature of the transducer 111 does not get excessively hot, which cooling may have the effect of improving the integrity and lifespan of the transducer 111. In certain embodiments, the cooling fluid stored in the reservoir 110 and/or the cartridge 130, and delivered from the reservoir 110 and/or the cartridge 130 to the balloon, is at room temperature. In other embodiments, the cooling fluid stored in the reservoir 110 and/or the cartridge 130 is cooled to below room temperature, so that the cooling fluid delivered from the reservoir 110 and/or the cartridge 130 to the balloon 112 is below room temperature, which should provide for better cooling compared to where the cooling fluid is at room temperature.

In certain embodiments, rather than the electrical cabling (e.g., 282, 482, 582, 682, 782, 882, or 982) being received within its own dedicated cable lumen (e.g., 326) of the catheter shaft 214, a same lumen of the catheter shaft can receive the electrical cabling (or at least a portion thereof) as well as at least a portion of the cooling fluid. For example, referring again to the example cross-sectional views of the catheter shaft 214 shown in FIGS. 3A1 and 3A2, one of the lumens 326, 327, 328 can receive the electrical cabling (or at least a portion thereof) as well as a portion of the cooling fluid. For a more specific example, the lumen 328 (if large enough) can receive the two separate coaxial cables 482a, 482b (that collectively comprise the electrical cabling 282) as well as the cooling fluid that is provided from the reservoir 110 and/or the cartridge 130 to the balloon 112 (or the cooling fluid that is returned from the balloon 112 to the reservoir 110 and/or the cartridge 130). Alternatively, one of the lumens 326, 327, 328 can receive the coaxial cable 482a (or 482b) and the cooling fluid that is provided from the reservoir 110 and/or the cartridge 130 to the balloon 112, and another one of the lumens 326, 327, 328 can receive the coaxial cable 482a (or 482b) and the cooling fluid that is returned from the balloon 112 the reservoir 110 and/or the cartridge 130. In such embodiments, the cooling fluid can also be used to cool the electrical cabling (or portion thereof) that is received within the same lumen as the cooling fluid, while the cooling fluid is also used to provide active cooling of about the first millimeter of tissue to thereby preserve the integrity of the blood vessel wall (e.g., the renal arterial wall), and to cool the transducer 111, so that the temperature of the transducer 111.

It is noted that the terms “cool” and “cooled”, as used herein, are used as relative terms, in that an element is considered to be cooled by a cooling fluid if a temperature of the element affected by the cooling fluid is less than the temperature the element would otherwise have if the element were not affected by the cooling fluid. For a specific example, a transducer submersed in a cooling fluid is considered to be “cooled” by the cooling fluid if a temperature of the transducer would be higher if the transducer were not submersed in the cooling fluid. For another example, electrical cabling that is received in a same lumen as cooling fluid is considered to be “cooled” by the cooling fluid if a temperature of the electrical cabling in a lumen that is devoid of the cooling fluid would be higher. For still another example, a blood vessel wall that is adjacent to a balloon (e.g., 112) including a transducer (e.g., 111) and filled with a cooling fluid is considered to be “cooled” by the cooling fluid if a temperature of the blood vessel wall would be higher in the absence of the cooling fluid. These are just a few examples, which are not intended to be all encompassing.

In certain embodiments, one of the lumens 326, 327, 328 can receive the electrical cabling 582, described with reference to FIG. 5, as well as the cooling fluid that is provided from the reservoir 110 and/or the cartridge 130 to the balloon 112 (or the cooling fluid that is returned from the balloon 112 to the reservoir 110 and/or the cartridge 130). In still another embodiment, one of the lumens 326, 327, 328 can receive the electrical cabling 682, described with reference to FIG. 6, as well as the cooling fluid that is provided from the reservoir 110 and/or the cartridge 130 to the balloon 112 (or the cooling fluid that is returned from the balloon 112 to the reservoir 110 and/or the cartridge 130).

Referring to FIGS. 3A1, 3A2, and 7, in a further embodiment, one of the lumens 326, 327, 328 can receive the electrical cabling 782a, as well as the cooling fluid that is provided from the reservoir 110 and/or the cartridge 130 to the balloon 112 (or the cooling fluid that is returned from the balloon 112 to the reservoir 110 and/or the cartridge 130); and another one of the lumens 326, 327, 328 can receive the electrical cabling 782b, as well as the cooling fluid that is returned from the balloon 112 to the reservoir 110 and/or the cartridge 130 (or the cooling fluid that is provided from the reservoir 110 and/or the cartridge 130 to the balloon 112). Referring to FIGS. 3A1, 3A2, and 8, in an embodiment, one of the lumens 326, 327, 328 can receive the electrical cabling 882, as well as the cooling fluid that is provided from the reservoir 110 and/or the cartridge 130 to the balloon 112 (or the cooling fluid that is returned from the balloon 112 to the reservoir 110 and/or the cartridge 130). Referring to FIGS. 3A1, 3A2, and 9, in an embodiment, one of the lumens 326, 327, 328 can receive the electrical cabling 982a, as well as the cooling fluid that is provided from the reservoir 110 and/or the cartridge 130 to the balloon 112 (or the cooling fluid that is returned from the balloon 112 to the reservoir 110 and/or the cartridge 130); and another one of the lumens 326, 327, 328 can receive the electrical cabling 982b, as well as the cooling fluid that is returned from the balloon 112 to the reservoir 110 (or the cooling fluid that is provided from the reservoir 110 and/or the cartridge 130 to the balloon 112). Referring to FIGS. 3A1, 3A2, and 10, in an embodiment, one of the lumens 326, 327, 328 can receive the electrical cabling 1082, as well as the cooling fluid that is provided from the reservoir 110 and/or the cartridge 130 to the balloon 112 (or the cooling fluid that is returned from the balloon 112 to the reservoir 110 and/or the cartridge 130).

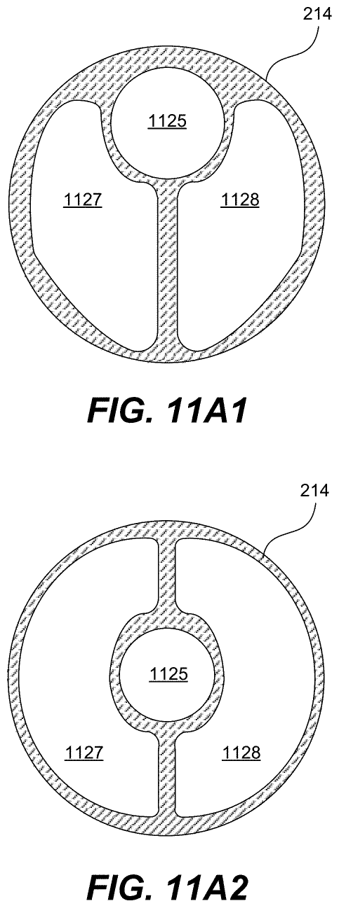

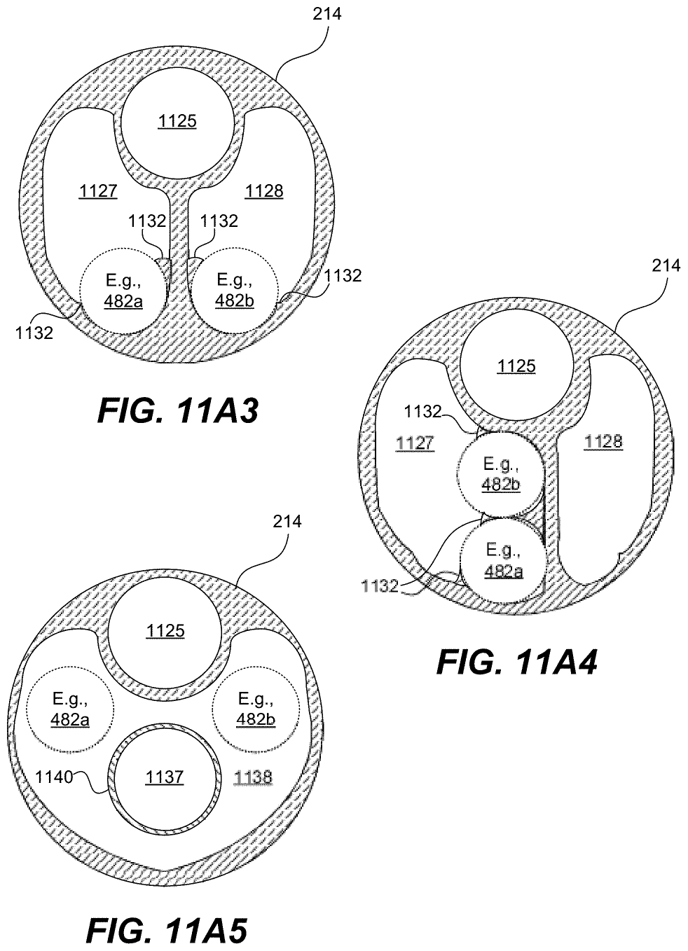

In each of the above-described embodiments, only two out of three of the lumens 326, 327, and 328 are utilized. Accordingly, in further embodiments one of the aforementioned lumens is eliminated, thereby enabling the cross-sectional area of one or more of the utilized lumens to be increased (as can be appreciated from FIGS. 11A1, 11A2, and 11A3 described below), and/or the overall cross-sectional area of the catheter shaft 214 to be reduced.

FIG. 11A1 illustrates a cross-sectional view of the catheter shaft 214, along the line A-A in FIG. 2C, in accordance with another embodiment of the present technology. In this embodiment, the catheter shaft includes a guidewire lumen 1125 offset from the center of the catheter shaft, and also includes lumens 1127 and 1128. FIG. 11A2 illustrates a cross-sectional view of the catheter shaft 214, along the line A-A in FIG. 2C, in accordance with still another embodiment of the present technology, where the guidewire lumen 1125 is located in the center of the catheter shaft 214 in order to center the transducer 111 within the catheter shaft 214. FIG. 11A3 illustrates a cross-sectional view of the catheter shaft 214, along the line A-A in FIG. 2C, in accordance with a further embodiment of the present technology.

Where electrical cabling or cable(s) is/are received within a same lumen that also receives cooling fluid, the cabling or cable(s) may float and/or freely move around within such a lumen. However, in such an embodiment it is possible that the cabling or cable(s) may move and/or bend in such a manner that the cabling or cable(s) occludes the lumen and adversely affects the flow of the cooling fluid between the balloon 112 and the reservoir 110 and/or the cartridge 130. To reduce the probability (and preferably prevent the possibility) of the cabling or cable(s) moving and/or bending in such a manner that the cabling or cable(s) occludes the lumen that also carries cooling fluid, one or more tabs, clips, clamps, or other types of connectors can hold the cabling or cable(s) in place within one or more of the lumens 1127, 1128. For example, in FIG. 11A3 the lumens 1127 and 1128 are shown as including tab type connectors 1132 that are used to hold or mount the cables (whose cross-sections are represented by the dashed lines) in place against the interior walls of the lumens 1127, 1128.

One of the lumens 1127, 1128 can receive the electrical cabling (or at least a portion thereof) as well as a portion of the cooling fluid. For a more specific example, the lumen 1128 (if large enough) can receive the two separate coaxial cables 482a, 482b (that collectively comprise the electrical cabling 282) as well as the cooling fluid that is provided from the reservoir 110 and/or the cartridge 130 to the balloon 112 (or the cooling fluid that is returned from the balloon 112 to the reservoir 110 and/or the cartridge 130). Alternatively, one of the lumens 1127, 1128 can receive the coaxial cable 482a (or 482b) and the cooling fluid that is provided from the reservoir 110 and/or the cartridge 130 to the balloon 112, and the other one of the lumens 1127, 1128 can receive the coaxial cable 482a (or 482b) and the cooling fluid that is returned from the balloon 112 the reservoir 110 and/or the cartridge 130.

In another embodiment, one of the lumens 1127, 1128 can receive the electrical cabling 582, described with reference to FIG. 5, as well as the cooling fluid that is provided from the reservoir 110 and/or the cartridge 130 to the balloon 112 (or the cooling fluid that is returned from the balloon 112 to the reservoir 110 and/or the cartridge 130). In still another embodiment, one of the lumens 1127, 1128 can receive the electrical cabling 682, described with reference to FIG. 6, as well as the cooling fluid that is provided from the reservoir 110 and/or the cartridge 130 to the balloon 112 (or the cooling fluid that is returned from the balloon 112 to the reservoir 110 and/or the cartridge 130).

Referring to FIGS. 11A1, 11A2, and 7, in a further embodiment, one of the lumens 1127, 1128 can receive the electrical cabling 782a, as well as the cooling fluid that is provided from the reservoir 110 and/or the cartridge 130 to the balloon 112 (or the cooling fluid that is returned from the balloon 112 to the reservoir 110 and/or the cartridge 130); and the other one of the lumens 1127, 1128 can receive the electrical cabling 782b, as well as the cooling fluid that is returned from the balloon 112 to the reservoir 110 and/or the cartridge 130 (or the cooling fluid that is provided from the reservoir 110 and/or the cartridge 130 to the balloon 112). Alternatively, one of the lumens 1127, 1128 can receive both of the cables 782a and 782b, in which case the other one of the lumens 1127, 1128 will be devoid of any electrical cabling.

Referring to FIGS. 11A1, 11A2, and 8, in an embodiment, one of the lumens 1127, 1128 can receive the electrical cabling 882, as well as the cooling fluid that is provided from the reservoir 110 and/or the cartridge 130 to the balloon 112 (or the cooling fluid that is returned from the balloon 112 to the reservoir 110 and/or the cartridge 130); while the other one of the lumens 1127, 1128 that is devoid of any electrical cabling is used to return the cooling fluid from the balloon 112 to the reservoir 110 and/or the cartridge 130 (or provides the cooling fluid from the reservoir 110 and/or the cartridge 130 to the balloon 112).

Referring to FIGS. 11A1, 11A2, and 9, in an embodiment, one of the lumens 1127, 1128 can receive the electrical cable 982a, as well as the cooling fluid that is provided from the reservoir 110 and/or the cartridge 130 to the balloon 112 (or the cooling fluid that is returned from the balloon 112 to the reservoir 110 and/or the cartridge 130); and the other one of the lumens 1127, 1128 can receive the electrical cable 982b, as well as the cooling fluid that is returned from the balloon 112 to the reservoir 110 and/or the cartridge 130 (or the cooling fluid that is provided from the reservoir 110 and/or the cartridge 130 to the balloon 112). Alternatively, one of the lumens 1127, 1128 can receive both of the cables 982a and 982b, in which case the other one of the lumens 1127, 1128 will be devoid of any electrical cabling.

Referring to FIGS. 11A1, 11A2, and 10, in an embodiment, one of the lumens 1127, 1128 can receive the electrical cabling 1082, as well as the cooling fluid that is provided from the reservoir 110 and/or the cartridge 130 to the balloon 112 (or the cooling fluid that is returned from the balloon 112 to the reservoir 110 and/or the cartridge 130); while other one of the lumens 1127, 1128 that is devoid of any electrical cabling returned from the balloon 112 is used to return the cooling fluid to the reservoir 110 and/or the cartridge 130 (or provides the cooling fluid from the reservoir 110 and/or the cartridge 130 to the balloon 112).

An advantage of the embodiments shown in FIGS. 11A1, 11A2, and 11A3 over the embodiments shown in FIGS. 3A1 and 3A2, is that each of the lumens 1127 and 1128 can have a significantly larger interior cross-sectional area than the lumens 326, 328, 327 due to a reduced number of lumens being included in the catheter shaft 214. This makes it easier for the electrical cabling (or at least a portion thereof) and cooling fluid (or at least a portion thereof) to share one or more common lumen(s).

In the embodiments shown in FIGS. 11A1, 11A2, and 11A3, the lumens 1127 and 1128 were shown as being mirror images of one another and having the same interior cross-sectional areas as one another and are presumed to have the same interior volumes as one another. Where electrical cabling or cable(s) is/are received within a lumen (e.g., 1127 and/or 1128) that also carries cooling fluid, the amount of interior cross-sectional area and interior volume that is available to carrying the cooling fluid will be reduced and may affect fluid dynamics. In order to provide for a constant and equal flow of cooling fluid from the reservoir 110 and/or the cartridge 130 to the balloon 112, and from the balloon 112 to the reservoir 110 and/or the cartridge 130, it is desirable that the interior cross-sectional area and the interior volume of the lumen (e.g., 1127) that is being used to transfer the cooling fluid from the reservoir 110 and/or the cartridge 130 to balloon 112 be substantially the same as the interior cross-sectional area and the interior volume of the lumen (e.g., 1128) that is being used to transfer the cooling fluid from the balloon 112 back to the reservoir 110 and/or the cartridge 130. This can be achieved using a pair of lumens (e.g., 1127 and 1128) that have the same interior cross-sectional areas as one another and the same interior volumes as one another, so long as each of the lumens receives a same sized portion of the cabling, e.g., where the lumen 1127 receives the cable 482a and the lumen 1128 receives the cable 482b; or where the lumen 1127 receives the cable 782a and the lumen 1128 receives the cable 782b; or where the lumen 1127 receives the cable 982a and the lumen 1128 receives the cable 982b.

FIG. 11A5 illustrates a cross-sectional view of the catheter shaft, along the line A-A in FIG. 2C, in accordance with still another embodiment of the present technology. In this embodiment, the catheter shaft includes a guidewire lumen 1125, as was also the case in the other embodiments described above. Additionally, the catheter shaft includes relatively large lumen 1138, which can be referred to as a larger lumen 1138. Within the larger lumen 1138 (which is shown as being generally heart shaped, but may have various other shapes) there is a tube 1140 that is free-floating, or alternatively attached to an interior wall of the larger lumen 1138 using one or more tabs, clips, clamps, or other types of connectors. The tube 1140 can be made of stainless steel, or plastic, but is not limited thereto. A hollow interior of the tube 1140 provides a smaller lumen 1137. The smaller lumen 1137 can be used to transfer cooling fluid from the reservoir 110 and/or the cartridge 130 to balloon 112, while the larger lumen 1138, which includes the cables 482a and 482b (or more generally, the electrical cabling) can be used to transfer the cooling fluid from the balloon 112 back to the reservoir 110 and/or the cartridge 130. Alternatively, the larger lumen 1138, which includes the electrical cabling, can be used to transfer cooling fluid from the reservoir 110 and/or the cartridge 130 to balloon 112, while the smaller lumen 1137 can be used to transfer the cooling fluid from the balloon 112 back to the reservoir 110 and/or the cartridge 130. The cables 482a and 482b (or more generally, the electrical cabling) can be free-floating within the larger lumen 1138, or alternatively, can be attached to the interior wall of the interior wall of the larger lumen 1138 using one or more tabs, clips, clamps, or other types of connectors.

In certain embodiments, e.g., wherein the inner conductors 450a, 450b are electrically coupled to the inner electrode 202, the other one the inner conductors 450a, 450b is electrically coupled to the outer electrode 203, and the outer tubular conducting shields 452a and 452b are commonly grounded, it may be advantageous to control the impedance of the cabling by configuring one of the fluid lumens of a pair of fluid lumens to receive all the cabling. However, where a pair of lumens (e.g., 1127 and 1128) have the same interior cross-sectional areas as one another and the same interior volumes as one another, if the electrical cabling or cables are received within one of the two lumens of the pair, then the one of the lumens (that receives the electrical cabling) will have significantly less interior cross-sectional area and interior volume available than the other to carry cooling fluid. To compensate or adjust for this, if the electrical cabling or cables is/are to be received within only one lumen of a pair of lumens, then the lumen that is to receive the cabling or cables (as well as carry cooling fluid) can be made larger than the other lumen that will carry cooling fluid while being devoid of any electrical cabling or cables. More specifically, the lumen that receive the electrical cabling can be made to have a greater interior cross-sectional area and a greater interior volume than the second lumen, which is devoid of the electrical cabling. Even more specifically, the lumen that receives the electrical cabling (as well as carries cooling fluid) can have a greater interior cross-sectional area and a greater interior volume than the other lumen (that will carry cooling fluid while being devoid of any electrical cabling), so that an available interior cross-sectional area and an available interior volume of the lumen (that receives the electrical cabling), which are available for providing the cooling fluid to the balloon or removing the cooling fluid from the balloon, are respectively substantially the same as an interior cross-sectional area and an interior volume of the other lumen (which is devoid of any electrical cabling). This way, the amount of the cooling fluid that is provided to the balloon 112 at any given time can be made substantially the same as the amount of cooling fluid that is removed from the balloon 112 at any given time. In such embodiments, the lumens 1127 and 1128 would be asymmetric, e.g., as shown in FIG. 11A4. For the purpose of the description herein, a first value or amount is considered to be substantially the same as a second value of amount where the first value is within +/−10% of the second value, or vice versa.

In an embodiment, the change in fluid dynamics caused by the change in the shape of the lumen 1127 containing both cables is also accounted for by changing the shape of the lumen 1128, as well as the interior cross-sectional area and the interior volume, such that the flow rates of the lumens 1127 and 1128 are equalized. In addition to the interior cross-sectional area and the interior volumes, the respective shapes of the fluid lumens may affect the fluid path/fluid dynamics of the system. By accounting for the change of shape in the lumen 1127 by changing the shape of the lumen 1128, as wells as by accounting for the change in interior cross-sectional area and interior volume, the flow rate of the two lumens 1127 and 1128 may be equalized, thereby maintaining a steady inflation of the balloon without the creation of hot pockets.

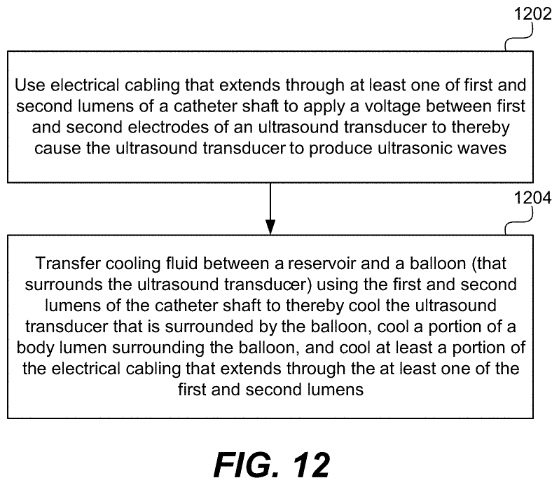

Embodiments of the present technology are also directed to various methods summarized with reference to the high-level flow diagram of FIG. 12. Certain such methods are for use with tissue treatment apparatus (e.g., 100) that comprises a catheter (e.g., 102) including a catheter shaft (e.g., 214) having a distal end and a proximal end, first and second lumens (e.g., 327 and 328, or 1127 and 1128) extending longitudinally through the catheter shaft between the distal and proximal ends thereof, an ultrasound transducer (e.g., 111) distally positioned relative to the distal end of the catheter shaft, and a balloon (e.g., 112) surrounding the ultrasound transducer. Referring to FIG. 12, such a method can include, at step 1202, using electrical cabling that extends through at least one of the first and the second lumens to apply a voltage between first and second electrodes (e.g., 202 and 203) of the ultrasound transducer to thereby cause the ultrasound transducer to produce ultrasonic waves. The method can also include, at step 1204, transferring a cooling fluid between a reservoir (e.g., 110) and/or the cartridge (e.g., 130) and the balloon (e.g., 112) using the first and the second lumens of the catheter shaft to thereby cool the ultrasound transducer that is surrounded by the balloon, and cool at least a portion of the electrical cabling that extends through at least one of the first and the second lumens. The cooling fluid can also be used to provide active cooling of about the first millimeter of body lumen tissue that is surrounding the balloon and transducer to thereby preserve the integrity of the blood vessel wall, e.g., the renal arterial wall. Additional details of how such a method can be achieved can be appreciated from the above discussion of the other FIGS.

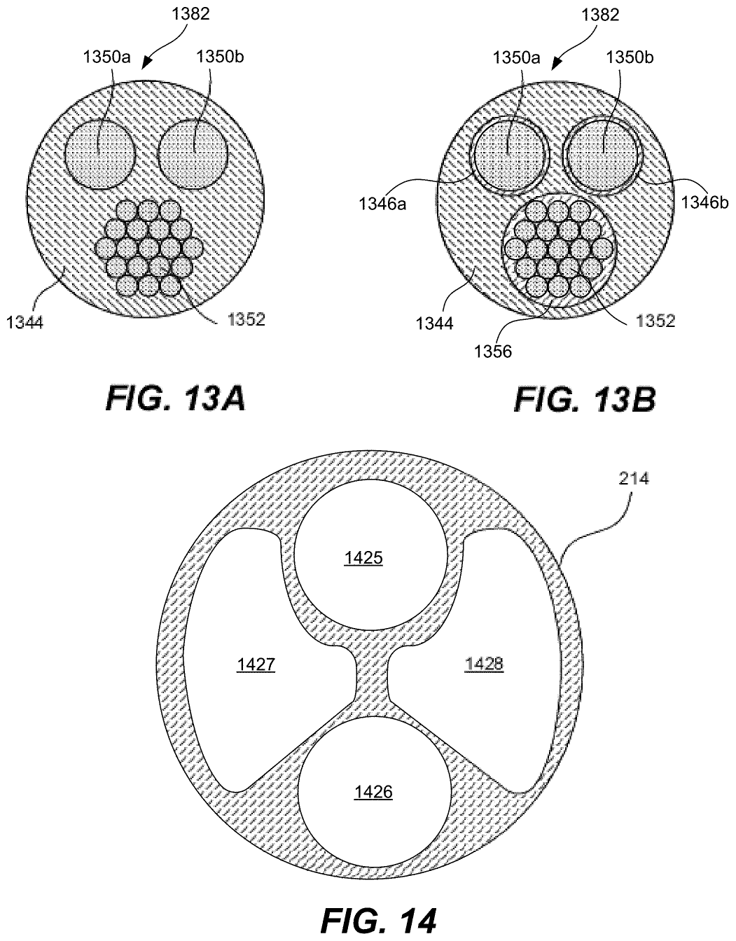

FIG. 13A illustrates a cross-sectional view of a single cable 1382 that includes a pair of solid wire inner conductors 1350a and 1350b and a stranded wire conductor 1352 that are surrounded by an insulating jacket 1344. The cable 1382 is another example of a cable that can be used to provide the electrical cabling 282 of the catheter 102 shown in FIG. 2A, in accordance with an embodiment. In an embodiment, the conductor wires 1350a, 1350b can each be, e.g., a 48 AWG non-stranded solid silver-plated copper alloy wire. FIG. 13B illustrates a cross-sectional view of the single cable 1382, according to another embodiment, where each of the conductors 1350a and 1350b is surrounded by its own respective insulation jacket 1346a and 1346b, and similarly, the conductor 1352 is also surrounded by its own insulation jacket 1356. The conductor wires 1350a and 1350b of the cable 1382 (in FIG. 13A or 13B) can be electrically coupled with outer electrode 203 on the proximal end of the transducer 211, e.g., by being soldered to the stepped portion 417 of the transducer 211. The conductor 1352 of the cable 1382 (in FIG. 13A or 13B) can be electrically coupled to the inner electrode 202 of the transducer 211, e.g., by being soldered to backing member 218, which is also known as the post 218. Inclusion of the insulation jackets 1346a and 1346b, shown in FIG. 13B, would reduce a probability that the wires 1350a and 1350b, which are coupled to the outer electrode 203 of the transducer 211, being inadvertently shorted to the post 218 of the transducer, which would undesirably result in a short circuit between the inner and outer electrodes 202 and 203 of the transducer 211. In an embodiment, the cable 1382 has a diameter of about 0.025 inches or smaller, and a cross-sectional area of about 0.0005 square inches or smaller. For example, if the diameter of the cable were 0.020 inches, the cross-sectional area would be about 0.00031 inches. It would also be possible for the conductor 1352 to be a solid wire conductor have a gauge in the range of 32 to 36 AWG, inclusive, rather than being a stranded wire conductor. It would also be possible for the conductors 1350a and 1350b to be stranded conductor wires, rather than solid conductors.

FIG. 14 illustrates a cross-sectional view of the catheter shaft 214, along the line A-A in FIG. 2C, in accordance with another embodiment of the present technology. In this embodiment, the catheter shaft includes a guidewire lumen 1425, a cable lumen 1426, and fluid lumens 1427 and 1428. The cable lumen 1426 in FIG. 14 is specifically designed to receive the electrical cabling 1382 shown in FIGS. 13A and 13B. Accordingly, the inner diameter of the cable lumen 1426 should be slightly larger than the outer diameter of the cable 1382 so that the cable lumen 1426 is capable of having the cable 1382 therein.

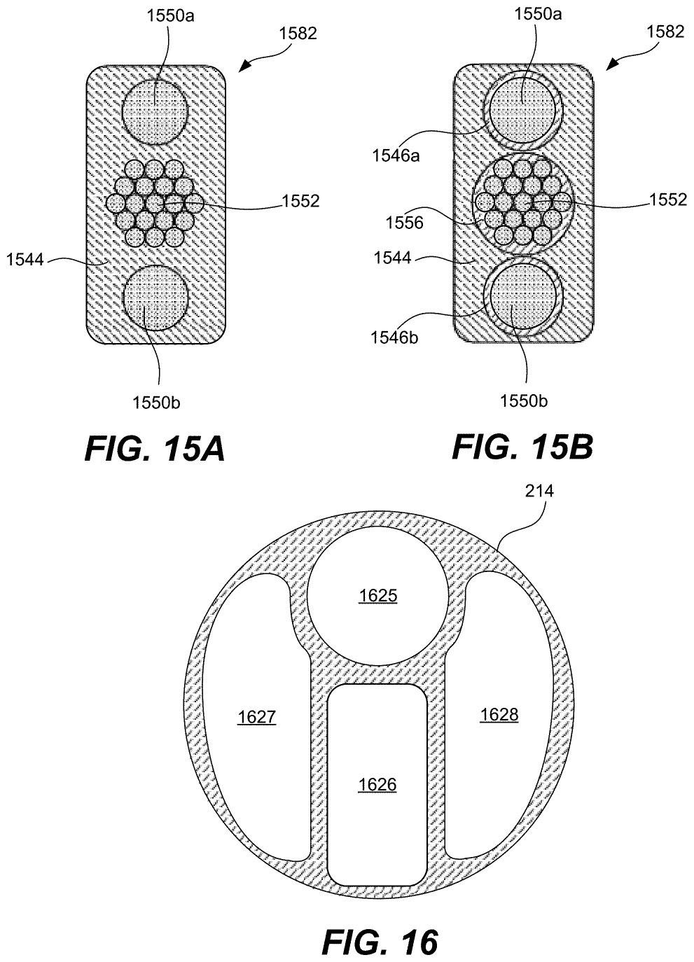

FIG. 15A illustrates a cross-sectional view of a single cable 1582 that includes a pair of solid wire inner conductors 1550a and 1550b and a stranded wire conductor 1552 that are surrounded by an insulating jacket 1544. The cable 1582 is another example of a cable that can be used to provide the electrical cabling 282 of the catheter 102 shown in FIG. 2A, in accordance with an embodiment. FIG. 15B illustrates a cross-sectional view of the single cable 1582, according to another embodiment, where each of the conductors 1550a and 1550b is surrounded by its own respective insulation jacket 1546a and 1546b, and similarly, the conductor 1552 is also surrounded by its own insulation jacket 1556. The conductor wires 1550a and 1550b of the cable 1582 can be electrically coupled with outer electrode 203 on the proximal end of the transducer 211, e.g., by being soldered to the stepped portion 417 of the transducer 211. The stranded wire conductor 1552 of the cable 1582 can be electrically coupled to the inner electrode 202 of the transducer 211, e.g., by being soldered to backing member 218, which is also known as the post 218. Inclusion of the insulation jackets 1546a and 1546b, shown in FIG. 15B, would reduce a probability that the wires 1550a and 1550b, which are coupled to the outer electrode 203 of the transducer 211, being inadvertently shorted to the post 218 of the transducer, which would undesirably result in a short circuit between the inner and outer electrodes 202 and 203 of the transducer 211. In an embodiment, the conductor wires 1550a, 1550b can each be, e.g., a 48 AWG non-stranded solid silver-plated copper alloy wire. In an embodiment, the cable 1582 has a width of 0.012 inches, a height of 0.025 inches, and a cross-sectional area of about 0.00030 square inches. It would also be possible for the conductor 1552 to be a solid wire conductor have a gauge in the range of 32 to 36 AWG, inclusive, rather than being a stranded wire conductor. It would also be possible for the conductors 1550a and 1550b to be stranded conductor wires, rather than solid conductors.

FIG. 16 illustrates a cross-sectional view of the catheter shaft 214, along the line A-A in FIG. 2C, in accordance with another embodiment of the present technology. In this embodiment, the catheter shaft includes a guidewire lumen 1625, a cable lumen 1626, and fluid lumens 1627 and 1628. The cable lumen 1626 in FIG. 16 is specifically designed to receive the electrical cabling 1682 shown in FIGS. 15A and 15B. Accordingly, the dimensions of the cable lumen 1526 should be slightly larger than the outer dimensions of the cable 1582 so that the cable lumen 1526 is capable of having the cable 1582 therein.

In certain embodiments, in order to sufficiently power the transducer 111 such that it emits sufficient unfocused ultrasound energy to heat tissue adjacent to a body lumen to ablate target nerves surrounding that body lumen, the collective current carrying capacity of the two conductors that are used to power the transducer 111 should be at least 0.8 Amps (A), and in certain embodiments, is about 1.0 A. Accordingly, where there are two conductors (e.g., 450a and 450b, 550a and 550b, 650a and 650b, 750a and 750b, 850a and 850b, 950a and 950b, 1050a and 1050b, 1350a and 1350b, or 1550a and 1550b) are connected to the same one of the electrodes (e.g., the outer electrode 203) of the transducer 111, and are used to provide power to the transducer 111, each of the two conductors should individually having a current carrying capacity of at least 0.4 A, and in certain embodiments a current carry capacity of about 0.5 A. The current carrying capacity of the remaining conductor, e.g., which is connected to the other one of the electrodes (e.g., the inner electrode 202) should also be at least 0.8 A, and in certain embodiments, is about 1.0 A. The term about, as used herein, means within +/−10% of a specified value.

Electrical Cabling Integrated with Catheter Shaft

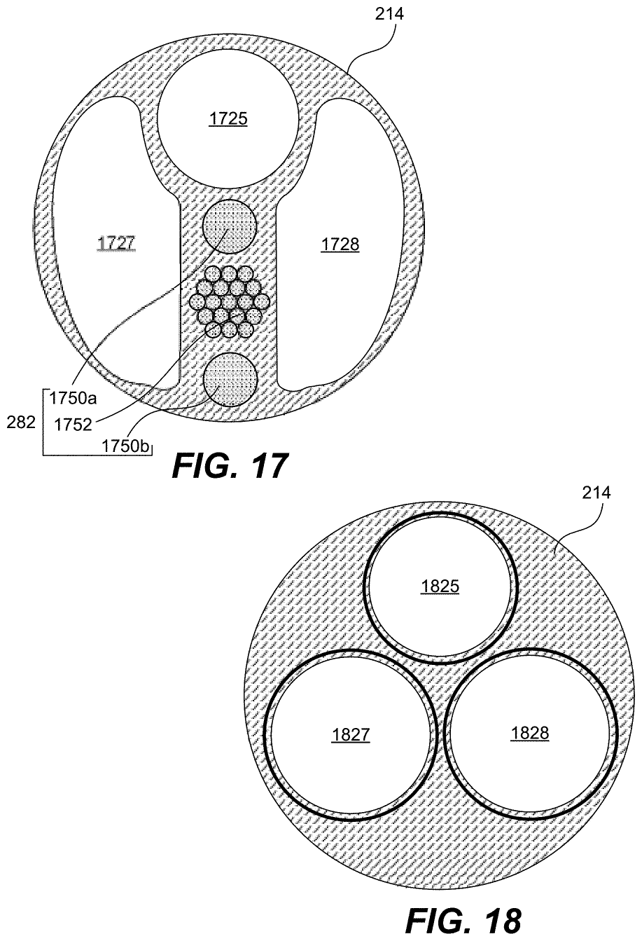

In the embodiments described above, the electrical cabling 282 used to apply a voltage between first and second electrodes (e.g., outer and inner electrodes) of the ultrasound transducer 111 (to thereby cause the ultrasound transducer 111 to generate ultrasonic waves) is manufactured separate from the catheter shaft 214, and thereafter, during assembly of the catheter 102 the electrical cabling 282 is threaded through a cable lumen of the catheter shaft 214, or alternatively, through one or two of the fluid lumens of the catheter shaft 214. In alternative embodiments, the electrical cabling is integrated into the catheter shaft itself when the catheter shaft is manufactured, e.g., such as when the catheter shaft is extruded. An example of such an embodiment is shown in FIG. 17, described below.

FIG. 17 illustrates a cross-sectional view of the catheter shaft 214, along the line A-A in FIG. 2C, in accordance with an embodiment of the present technology where the electrical cabling 282 is integrated into the catheter shaft. 214. In FIG. 17, the catheter shaft includes a guidewire lumen 1725, and fluid lumens 1827 and 1828. In FIG. 17, the catheter shaft does not include (i.e., is devoid of) a cable lumen. Rather, the conductors 1750a, 1750b, and 1752 are integrated into the catheter shaft itself during manufacture of the catheter shaft. In FIG. 17, the conductors 1750a and 1750b are each shows as being a solid wire, and the conductor 1752 is shown as being a stranded wire. In an embodiment, the conductor wires 1750a, 1750b can each be, e.g., a 48 AWG non-stranded solid silver-plated copper alloy wire. The conductors 1750a and 1750b can be electrically coupled with outer electrode 203 on the proximal end of the transducer 211, e.g., by being soldered to the stepped portion 417 of the transducer 211. The conductor 1752 can be electrically coupled to the inner electrode 202 of the transducer 211, e.g., by being soldered to backing member 218, which is also known as the post 218. It would also be possible for the conductor 1752 to be a solid wire conductor have a gauge in the range of 32 to 36 AWG, inclusive, rather than being a stranded wire conductor. It would also be possible for the conductors 1750a and 1750b to be stranded conductor wires, rather than solid conductors. A benefit of having the cabling integrated into the catheter shaft is that less cross-sectional area of the catheter shaft is occupied by the cabling, thereby allowing the size of the fluid lumens to be increased (compared to if the catheter shaft includes a cable lumen), and/or the overall size (i.e., gauge) of the catheter shaft can be reduced.

In the embodiment shown in FIG. 17, the various conductors of the electrical cabling 282 were shown as extended generally parallel to one another generally through a central portion of the catheter shaft 214. In an alternative embodiment, such as in the embodiment shown in FIG. 18, one or more of the conductors of the electrical cabling 282 can spiral around one or more of the lumens that extend longitudinally through the catheter shaft 214, such as around a cable lumen 1825, around a fluid lumen 1827, and/or around a fluid lumen 1828. In FIG. 18, the thick circles surrounding the lumens 1825, 1827 and 1828 are examples of where such spiral conductors of the electrical cabling may be located. Different conductors of the electrical cabling can spiral around different lumen. Alternatively, two or more conductors can spiral around a same lumen, in which case the multiple conductors should be electrically isolated from one another. Alternatively, or additionally, one or more conductors of the electrical cabling can spiral about a portion of the catheter body close to an outer periphery of the catheter body, e.g., within about 0.1 inches of the outer periphery of the catheter body.

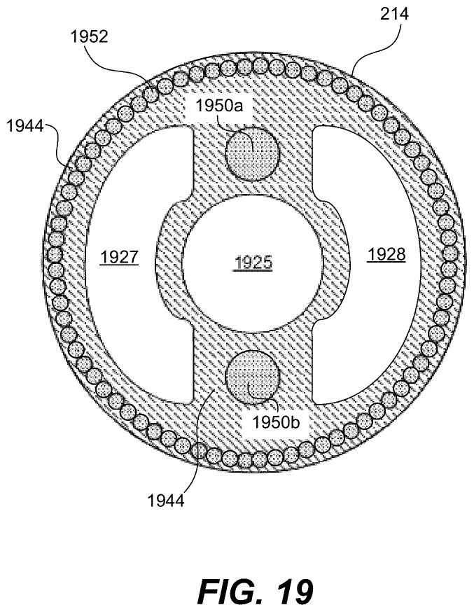

FIG. 19 illustrates a cross-sectional view of the catheter shaft 214, along the line A-A in FIG. 2C, in accordance with another embodiment of the present technology where the electrical cabling 282 is integrated into the catheter shaft. 214. In FIG. 19, the catheter shaft includes a guidewire lumen 1925, and fluid lumens 1927 and 1928, but does not include (i.e., is devoid of) a cable lumen. Rather, the conductors 1950a, 1950b, and 1952 are integrated into the catheter shaft 214 itself during manufacture of the catheter shaft. In FIG. 19, the conductors 1950a and 1950b, which can also be referred to as inner conductors, are each shown as being a solid wire. In certain embodiments, the inner conductors 1950a, 1950b each comprise one wire constructed, e.g., of 42 AWG (American Wire Gauge) solid silver-plated copper alloy. It would also be possible to replace one or both of the inner conductors 1950a, 1950b with stranded conductors. The conductors 1952 comprise an outer tubular conducting shield, which can be constructed, e.g., of a plurality of 48 AWG silver-plated copper wires, and can be surrounded by an insulating jacket 1944. Similarly, the inner conductors 1950a, 1950b can be surrounded by a dielectric 1944. The insulating jacket 1944 and the dielectric 1944 can be made of a non-electrically conductive material, such as, but are not limited, thermoplastic elastomers (such as those marked under the trademark PEBAX™), medical-grade thermoplastic polyurethane elastomers (such as those marketed under the trademark PELLETHANE™), pellethane, isothane, or other suitable polymers or any combination thereof. The conductors 1950a and 1950b can be electrically coupled with outer electrode 203 on the proximal end of the transducer 211, e.g., by being soldered to the stepped portion 417 of the transducer 211. Wires of outer tubular conducting shield 1952 can be bundled together and soldered to the backing member 218 (which can also be referred to as the post 218) to thereby electrically couple the outer tubular conducting shield to the inner electrode 202 of the transducer 211.

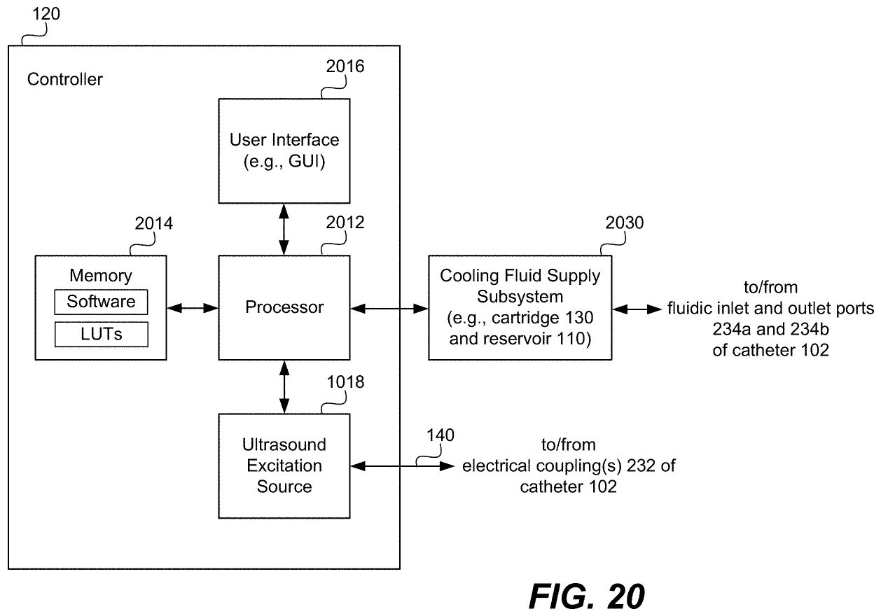

FIG. 20 will now be used to describe an example implementation of the controller 120, which was introduced in FIG. 1. Referring to FIG. 20, the controller 120 is shown as including a processor 2012, memory 2014, a user interface 2016, and an ultrasound excitation source 2018, but can include additional and/or alternative components. While not specifically shown, the processor 2012 can be located on a control board, or more generally, a printed circuit board (PCB) along with additional circuitry of the controller 120. The user interface 2016 interacts with the processor 2012 to cause transmission of electrical signals at selected actuation frequencies to the ultrasound transducer 111 via wires of the connection cable 140 and the cabling 282 that extends through the catheter shaft 112. These wires electrically couple the controller 120 to the transducer 111 so that the controller 120 can send electrical signals to the transducer 111, and receive electrical signals from the transducer 111. The processor 2012 can control the ultrasound source 2018 to control the amplitude and timing of the electrical signals so as to control the power level and duration of the ultrasound signals emitted by transducer 111. More generally, the controller 120 can control one or more ultrasound treatment parameters that are used to perform sonication. In certain embodiments, the excitation source 2018 can also detect electrical signals generated by transducer 111 and communicate such signals to the processor 2012 and/or circuitry of a control board. While the ultrasound excitation source 2018 in FIG. 20 is shown as being part of the controller, it is also possible that the ultrasound excitation source 2018 is external to the controller 120 while still being controller by the controller 120, and more specifically, by the processor 2012 of the controller 120. The ultrasound excitation source 2018 can also be referred to as a signal generator 2018.

The user interface 2016 can include a touch screen and/or buttons, switches, etc., to allow for an operator (aka user) to enter patient data, select a treatment parameters, view records stored on a storage/retrieval unit (not shown), and/or otherwise communicate with the processor 2012. The user interface 2016 can include a voice-activated mechanism to enter patient data or may be able to communicate with additional equipment so that control of the controller 120 is through a separate user interface, such as a wired or wireless remote control. In some embodiments, the user interface 2016 is configured to receive operator-defined inputs, which can include, e.g., a duration of energy delivery, one or more other timing aspects of the energy delivery pulses (e.g., frequency, duty cycle, etc.), power, and/or mode of operation, or a combination thereof. Example modes of operation can include (but are not limited to): system initiation and set-up, catheter preparation, balloon inflation, verification of balloon apposition, pre-cooling, sonication, post-cooling, balloon deflation, and catheter removal, but are not limited thereto. In certain embodiments, the user interface 2016 provides a graphical user interface (GUI) that instructs a user how to properly operate the system 100. The user interface 2016 can also be used to display treatment data for review and/or download, as well as to allow for software updates, and/or the like.

The controller 120 can also control a cooling fluid supply subsystem 2030, which can include the cartridge 130 and reservoir 110, which were described above with reference to FIGS. 1 and 3C, but can include alternative types of fluid pumps, and/or the like. The cooling fluid supply subsystem 2030 is fluidically coupled to one or more fluid lumens (e.g., 327 and 328) within catheter shaft 214 which in turn are fluidically coupled to the balloon 112. The cooling fluid supply subsystem 2030 can be configured to circulate a cooling liquid through the catheter 102 to the transducer 111 in the balloon 112. The cooling fluid supply subsystem 2030 may include elements such as a reservoir 110 for holding the cooling fluid 213, pumps (e.g., syringes 342a and 342b), a refrigerating coil (not shown), or the like for providing a supply of cooling fluid to the interior space of the balloon 112 at a controlled temperature, desirably at or below body temperature. The processor 2012 interfaces with the cooling fluid supply subsystem 2030 to control the flow of cooling fluid into and out of the balloon 112. For example, the processor 2012 can control motor control devices linked to drive motors associated with pumps for controlling the speed of operation of pumps (e.g., syringes 342a, 342b). Such motor control devices can be used, for example, where the pumps are positive displacement pumps, such as peristaltic pumps. Alternatively, or additionally, a control circuit may include structures such as controllable valves connected in the fluid circuit for varying resistance of the circuit to fluid flow (not shown). The processor 2012 can monitor pressure measurements obtained by the pressure sensors to monitor and control the cooling fluid through the catheter 102 and the balloon 112. The pressure sensors can also be used to determine if there is a blockage and/or a leak in the catheter 102. While the balloon 112 is in an inflated state, the pressure sensors can be used maintain a desired pressure in the balloon 112, e.g., at a pressure of 10 psi, but not limited thereto.

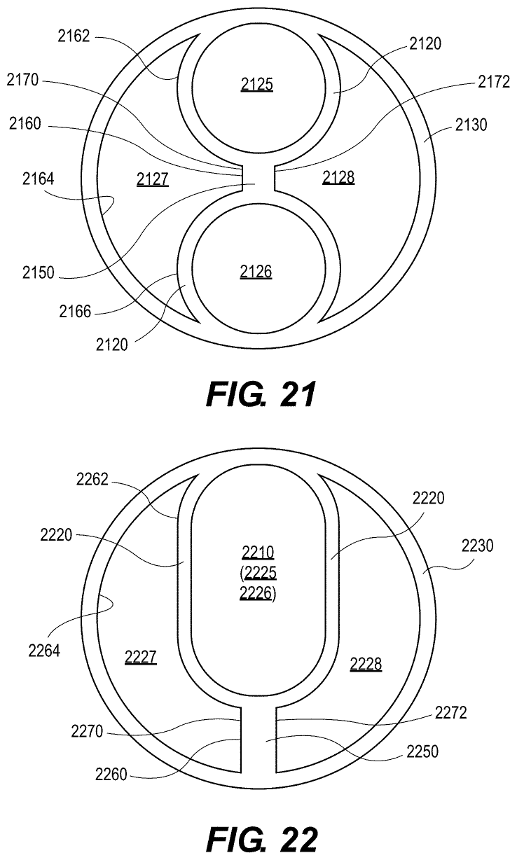

Referring to FIG. 21, a cross-sectional view of a catheter shaft, along the line A-A in FIG. 2C, is shown in accordance with another embodiment of the present technology. In this embodiment, the catheter shaft includes a guidewire lumen 2125, a cable lumen 2126, and fluid lumens 2127 and 2128.

The guidewire lumen 2125 and the cable lumen 2126 may have circular profiles. The circular profiles can be defined within annular lumen walls 2120 containing the respective lumens 2125, 2126. The annular lumen wall 2120 containing the guidewire lumen 2125 may be referred to as a guidewire lumen wall, and the annular lumen wall 2120 containing the cable lumen 2126 may be referred to as a cable lumen wall. Accordingly, each lumen can have a diameter, and the diameter may be sized according to the element that is received by the lumen. For example, the guidewire lumen 2125 may be sized to receive a guidewire in a sliding fit. Accordingly, the guidewire lumen 2125 may have a lumen diameter in a range of about 0.019 to 0.023 inch, e.g., 0.021 inch. The cable lumen 2126 can receive the electrical cabling described above, e.g., electrical cabling 1382 shown in FIGS. 13A and 13B. Accordingly, the inner diameter of the cable lumen 2126 may be slightly larger than the outer diameter of the electrical cabling so that the cable lumen 2126 is capable of having the electrical cabling therein. For example, the cable lumen 2126 may have a lumen diameter in a range of about 0.019 to 0.023 inch, e.g., 0.021 inch. In an embodiment, the guidewire lumen 2125 and the cable lumen 2126 have a same size and shape, however, the lumens may alternatively have different sizes and/or shapes.

Whereas the guidewire lumen 2125 and the cable lumen 2126 may be sized to fit their respective payloads, an outer wall 2130 can be sized and shaped to achieve an optimal size and robustness of the catheter. For example, as described above, radial access may benefit from a catheter shaft having an outer diameter of 5 French or less, e.g., 5 French to 4 French. Accordingly, the outer wall 2130 may have an annular shape, encompassing the guidewire lumen 2125 and cable lumen 2126. The outer wall 2130 may have an outer diameter in a range of about 0.053 to 0.066 inch, e.g., 0.057 inch. The outer wall 2130 may also have an outer wall thickness that provides for robust resistance to expansion and/or collapse in response to pressure changes within the fluid lumens 2127, 2128. It has been discovered that an outer wall 2130 having an outer wall thickness in a range of about 0.002 to 0.003 inch, e.g., between 0.0020 to 0.0030 inch, provides sufficient wall strength. For example, an outer wall thickness within that range, e.g., 0.0025 inch, can resist undue expansion or collapse under the fluid flow rates used for radial access procedures, as described above.

The catheter shaft can incorporate a stiffening web 2150 to support the fluid lumen walls and resist collapse of the fluid lumens walls when pressure changes within the fluid lumens 2127, 2128. The stiffening web 2150 can have a stiffening wall thickness, measured between the adjacent inner walls 2170, 2172 of the fluid lumen 2127 and the fluid lumen 2128, which is greater than a thickness of the outer wall 2130 and/or the lumen walls surrounding the guidewire lumen 2125 and the cable lumen 2126. For example, the outer wall 2130 can have an outer wall thickness of 0.0025 inch, however, the stiffening web 2150 can have a stiffening wall thickness in a range of about 0.003 to 0.007 inch, e.g., 0.005 inch. Accordingly, in an embodiment, the stiffening wall thickness is at least twice the outer wall thickness. Although the cross-sectional area of the fluid lumens 2127, 2128 could be increased by providing a consistent wall thickness for all walls of the cross-sectional profile, in an embodiment the stiffening web 2150 is thicker at the expense of reduced lumen area in order to stiffen and support the lumen walls during use.

Additional features that may be incorporated into the cross-sectional profile, even at the expense of reduced lumen area, can include fillets or chamfers at each of the internal edges of the fluid lumens 2127, 2128. By way of example, an edge at which an inner surface of the outer wall meets an outer surface of the guidewire lumen wall of the guidewire lumen 2125, an edge at which the inner surface of the outer wall meets an outer surface of the cable lumen wall of the cable lumen 2126, and/or an edge at which the outer surface of the stiffening web 2150 meets an outer surface of either of the lumen walls, can have a radius. The rounded or chamfered internal edges can provide a stable structure that may be more easily manufactured because pins used during the forming process may be more readily removed from the extruded catheter shaft.

A cross-sectional area of the fluid lumens 2127, 2128 can be maximized, while taking the sizes and shapes of the guidewire lumen 2125, the cable lumen 2126, and the outer wall 2130, into account. More particularly, each of the fluid lumens 2127, 2128 can have a minimum cross-sectional area of about 0.00055 square inch. For example, each fluid lumen can have a cross-sectional area in a range of 0.00055 to 0.00059 square inch, e.g., 0.00057 square inch, to achieve a targeted head pressure used in a radial access procedure. The head pressure target may be, for example, 34 psi or less when a length of the fluid lumens 2127, 2128 is about 145 cm (about 290 cm total flow path length in the case of two fluid lumens) for use in radial access procedures using a catheter having a catheter shaft that is 5F or less in diameter. The head pressure target may be, for example, 34 psi or less when a length of the fluid lumens 2127, 2128 is at least 145 cm (at least about 290 cm total flow path length in the case of two fluid lumens) for use with a catheter shaft having an outer diameter of 5F or less. The total flow path can be based on an overall working length of the catheter used to reach a distal end of a renal artery via a radial access approach. For example, the overall working length may be in a range of 145 to 175 cm, and thus, each fluid lumen can have a similar length, resulting in a total flow path length in a range of 290 cm to 350 cm. In an embodiment, the overall working length of the catheter is in a range of 144 to 146 cm, e.g., 145 cm, and thus, the total flow path length can be in a range of 288 cm to 292 cm, e.g., 290 cm. In an embodiment, however, the overall working length of the catheter (and a length of each fluid lumen) is a minimum of 145 cm.

The shape of the fluid lumens 2127, 2128 can be defined by the surfaces of the adjoining shaft sectional areas that bound them. More particularly, each fluid lumen may be bounded by a surface of the lumen wall surrounding the guidewire lumen 2125, the lumen wall surrounding the cable lumen 2126, the stiffening web 2150, and the outer wall 2130. The surfaces adjoin each other to surround the respective fluid lumen.

Profiles of the adjoining surfaces circumscribe the fluid lumens 2127, 2128. A profile of the adjoining surfaces circumscribing the fluid lumen 2127 is now described. It will be appreciated that the profile circumscribing the fluid lumen 2128 may be a mirror image, symmetric about a vertical plane extending vertically through a middle of the stiffening web and dividing the fluid lumens 2127, 2128. The mirrored design of the catheter shaft profile can allow for a proximal bifurcation to be easily formed between the fluid lumens, and may contribute to an easier extrusion process during shaft manufacturing.

In an embodiment, the stiffening web 2150 has a web surface 2160 extending vertically from an inner edge at the guidewire lumen 2125 to an inner edge at the cable lumen 2126. More particularly, the web surface 2160 can have a web length extending from a first web end at that guidewire lumen wall to a second web end at the cable lumen wall.

A guidewire lumen surface 2162 can arc outward from the first web end. More particularly, the guidewire lumen surface 2162 can arc from a first arc end at the web surface 2160 to a second arc end at a shaft wall surface 2164. The shaft wall surface 2164 can be an inner surface of the outer wall 2130. The arc of the guidewire lumen surface 2162 can have a radius from a centerpoint of the guidewire lumen 2125. Accordingly, the inner edges at the web surface 2160 and the shaft wall surface 2164 can be at a same distance from the centerpoint of the guidewire lumen 2125.

A cable lumen surface 2166 can arc outward from the second web end. More particularly, the cable lumen surface 2166 can arc from a first arc end at the web surface 2160 to a second arc end at the shaft wall surface 2164. The arc of the cable lumen surface 2166 can have a radius from a centerpoint of the cable lumen 2126. Accordingly, the inner edges at the web surface 2160 and the shaft wall surface 2164 can be at a same distance from the centerpoint of the cable lumen 2126.

The shaft wall surface 2164 can arc between the guidewire lumen surface 2162 and the cable lumen surface 2166. More particularly, the arc of the shaft wall surface 2164 can have a radius from a center point of the catheter shaft, e.g., at a middle of the stiffening web 2150. The arc of the shaft wall surface 2164 can be concave inward toward the fluid lumen 2127, whereas the arcs of the guidewire lumen surface 2162 and the cable lumen surface 2166 can be concave outward toward the fluid lumen 2127. The fluid lumen 2127 (and mirrored fluid lumen 2128) can have sufficient area for radial access flow requirements and can be provided in a compact catheter size.

Referring to FIG. 22, a cross-sectional view of a catheter shaft, along the line A-A in FIG. 2C, is shown in accordance with another embodiment of the present technology. In this embodiment, the catheter shaft includes a guidewire lumen 2225 and a cable lumen 2226, both of which can occupy a same space. More particularly, a hybrid lumen 2210 can be provided that includes a portion to receive a guidewire (guidewire lumen 2225) and a portion to receive the electrical cabling (cable lumen 2226). The catheter shaft can include fluid lumens 2227 and 2228.

The hybrid lumen 2210 can have an oval profile. The oval profile can be defined within annular lumen walls 2220 containing the hybrid lumen 2210. The hybrid lumen 2210 can have a width between apposing vertical walls, and the walls can terminate at rounded ends having respective radii. Accordingly, the hybrid lumen can be sized to receive one or more of the guidewire or the electrical cabling.

An outer wall 2230 can be sized and shaped to achieve an optimal size and robustness of the catheter. For example, as described above, radial access may benefit from a catheter shaft having an outer diameter of 5 French to 4 French. Accordingly, the outer wall 2230 may have an annular shape, encompassing the hybrid lumen 2210, and the annular wall may have an outer diameter in a range of about 0.053 to 0.066 inch, e.g., 0.057 inch. The outer wall 2230 may also have an outer wall thickness that provides for robust resistance to expansion and/or collapse in response to pressure changes within the fluid lumens 2227, 2228. It has been discovered that an outer wall thickness in a range of about 0.002 to 0.003 inch, e.g., between 0.0020 to 0.0030 inch provides sufficient wall strength. For example, an outer wall thickness within that range, e.g., 0.0025 inch, resists undue expansion or collapse under the fluid flow rates used for radial access procedures, as described above.

The catheter shaft can incorporate a stiffening web 2250 to support the fluid lumen walls and resist collapse of the fluid lumens walls when pressure changes within the fluid lumens 2227, 2228. The stiffening web 2250 can have a stiffening wall thickness, measured between the adjacent inner walls 2270, 2272 of the fluid lumen 2227 and the fluid lumen 2228, which is greater than a thickness of the outer wall 2230 and/or the lumen walls surrounding the hybrid lumen 2210. For example, the outer wall 2230 can have an outer wall thickness of about 0.0025 inch, however, the stiffening web 2250 can have a stiffening wall thickness in a range of about 0.003 to 0.007 inch, e.g., 0.005 inch. Accordingly, in an embodiment, the stiffening wall thickness is at least twice the outer wall thickness. Although the cross-sectional area of the fluid lumens 2227, 2228 could be increased by providing a consistent wall thickness for all walls of the cross-sectional profile, in an embodiment the stiffening web 2250 is thicker at the expense of reduced lumen area in order to stiffen and support the lumen walls during use. As described above with respect to FIG. 21, the catheter shaft of FIG. 22 may also include fillets or chamfers at each of the internal edges of the fluid lumens 2227, 2228.

A cross-sectional area of the fluid lumens 2227, 2228 can be maximized, while taking the sizes and shapes of the hybrid lumen 2210 and the outer wall 2230, into account. More particularly, each of the fluid lumens 2227, 2228 can have a minimum cross-sectional area of about 0.00055 square inch. For example, each fluid lumen can have a cross-sectional area in a range of 0.00055 to 0.00059 square inch, e.g., 0.00058 square inch, to achieve a head pressure target used in a radial access procedure. The head pressure target may be, for example, about 34 psi or less when a length of each of the flow lumens 2227, 2228 is about 145 cm (about 290 cm total flow path length in the case of two fluid lumens) for use in radial access procedures. The total flow path can be based on an overall length of the catheter used to reach a distal end of a renal artery via a radial access approach. For example, the overall working length may be in a range of 145 to 175 cm, and thus, each fluid lumen can have a similar length, resulting in a total flow path length in a range of 290 to 350 cm. In an embodiment, the overall working length of the catheter is in a range of 144 to 146 cm, e.g., 145 cm, and thus, the total flow path length can be in a range of 288 to 292 cm, e.g., 290 cm. In an embodiment, however, the overall working length of the catheter (and a length of each fluid lumen) is a minimum of 145 cm.

The shape of the fluid lumens 2127, 2128 can be defined by the surfaces of the adjoining shaft sectional areas that bound them. More particularly, each fluid lumen may be bounded by a surface of the lumen wall 2220 surrounding the hybrid lumen 2210, the stiffening web 2250, and the outer wall 2230. The surfaces adjoin each other to surround the respective fluid lumen.

Profiles of the adjoining surfaces circumscribe the fluid lumens 2227, 2228. A profile of the adjoining surfaces circumscribing the fluid lumen 2227 is now described. It will be appreciated that the profile circumscribing the fluid lumen 2228 may be a mirror image, symmetric about a vertical plane extending vertically through a middle of the stiffening web 2250 and dividing the fluid lumens 2227, 2228. The mirrored design of the catheter shaft profile can allow for a proximal bifurcation to be easily formed between the fluid lumens, and may contribute to an easier extrusion process during shaft manufacturing.

In an embodiment, the stiffening web 2250 has a web surface 2260 extending vertically from an inner edge at the hybrid lumen 2210 to an inner edge at the catheter wall 2230. More particularly, the web surface 2260 can have a web length extending from a first web end at that guidewire lumen 2210 to a second web end at the outer wall 2230.

A hybrid lumen surface 2262 can arc outward from the first web end. More particularly, the hybrid lumen surface 2262 can arc from a first arc end at the web surface 2260 to a second arc end at a shaft wall surface 2264. The shaft wall surface 2264 can be an inner surface of the outer wall 2230. The hybrid lumen surface 2262 can extend along a path that conforms to the hybrid lumen wall 2220. For example, the hybrid lumen surface can be a portion of an oval profile.

The shaft wall surface 2264 can arc between the hybrid lumen surface 2262 and the web surface 2260. More particularly, the arc of the shaft wall surface 2264 can have a radius from a center point of the catheter shaft, e.g., at a centerpoint around which the outer surface of the outer wall 2230 extends. The arc of the shaft wall surface 2264 can be concave inward toward the fluid lumen 2227, whereas the hybrid lumen surface 2262 and the web surface 2260 can face outward toward the fluid lumen 2227. The fluid lumen 2227 (and mirrored fluid lumen 2228) can have sufficient area for radial access flow requirements and can be provided in a compact catheter size.

The catheter shaft configurations of FIGS. 21-22 can achieve the same technical advantages described above, e.g., realizing faster and less painful procedures that result in fewer complications. Additionally, the size and shape of the fluid lumens can permit optimal fluid dynamics to enable fluid flow rates that are used for a radial access catheter. More particularly, the catheter shaft allows the catheter size to decrease, while maintaining the fluid flow rates that are needed for radial access procedures.

There are various different ways that that conductors of the electrical cabling can be integrated into the catheter shaft itself. In certain embodiments, a laminated shaft construction method can be used to make the catheter shaft, which involves bundling components inside a single lumen thermoplastic tube, using mandrels to create thru-lumens, and then using an outer heat shrink and vertical laminator to reflow all the components together. The mandrels and heat shrink could thereafter be removed one or more later steps. This method can be used to include embedded conductors that are linear or in a spiraled (also known as coiled) configuration. Other variations are also possible and within the scope of the embodiments described herein.

Where various conductors of the electrical cabling integrated into the catheter shaft itself, portions of the catheter shaft (that are not the conductors of the electrical cabling) should be made of a non-electrically conductive material so that the non-electrically conductive material can provide insulation for the various conductors, to electrically isolate the conductors of the electrical cabling from one another. Examples of non-electrically conductive material from which portions of the catheter shaft (that are not the conductors of the electrical cabling) can be made include, but are not limited, thermoplastic elastomers (such as those marked under the trademark PEBAX™), medical-grade thermoplastic polyurethane elastomers (such as those marketed under the trademark PELLETHANE™), pellethane, isothane, or other suitable polymers or any combination thereof.

Combinations of the above described embodiments are also possible and within the scope of the embodiments described herein. For example, one or more conductors of the electrical cabling that is/are integrated into the catheter shaft can extend longitudinally through the catheter shaft generally parallel to the various lumen, and one or more further conductors of the electrical cabling can spiral around one or more of the lumen and/or can spiral about a portion of the catheter body close to an outer periphery of the catheter body, e.g., within about 0.1 inches of the outer periphery of the catheter body.

Embodiments of the present technology that enable the catheter shaft 214 to be reduced in size, e.g., from having a 6 French diameter to having a 5 French diameter, or even a 4 French diameter, provide for improved radial access to the renal arteries, wherein the smaller the diameter the better it is to provide for easier and less painful access to the renal arteries. The improved access may also reduce a likelihood of complications and facilitate faster procedure and recovery times. Certain techniques for reducing the diameter of the catheter shaft 214 that were described above include using a single electrical cable rather than a pair of cables, which enables the cable lumen to be reduced in size. Another technique for reducing the diameter of the catheter shaft is to not include a cable lumen that is dedicated to holding electrical cabling, i.e., to make the catheter shaft 214 devoid of a cable lumen. One way to make the catheter shaft 214 devoid of a cable lumen is to include the electrical cabling within one or both of the fluid lumen of the catheter shaft, e.g., as was described above with reference to FIGS. 11A3, 11A4, 11A5, and 12. Another way to make the catheter shaft 214 devoid of a cable lumen is to integrate the conductors of the electrical cabling into the catheter shaft itself, e.g., as described above with reference to FIGS. 17-19.

This invention will help enable a smaller catheter (going from 6F to 5F, and even 4F or 3F). In order to get radial access, a smaller catheter is required. The groin is a more painful route to take. The cable needs to be suitable to carry the large amount of power necessary to denervate nerves, while conserving real estate in the catheter so as not to disturb the fluid dynamics/cooling effect (which effects energy delivery—both far field and near field) necessary to provide a safe and effective ablation.

The transducers, apparatuses, systems, and methods described herein may be used to treat any suitable tissue, which tissue may be referred to as a target anatomical structure. For example, use of the present systems to treat (e.g., neuromodulate) the renal nerve is described above. It should be appreciated that body lumens, in which the present systems may be positioned for treating tissue, are not necessarily limited to naturally occurring body lumens. For example, the treatment may include creating a body lumen within tissue (e.g., using drilling, a cannula, laser ablation, or the like) and then positioning suitable components within such a body lumen. Other suitable applications for the present system include ablation of pulmonary nerve and tissue responsible veins or cardiac arrhythmia, nerves within that intervertebral disk, nerves within or outside of that intervertebral disk, basivertebral nerves within that vertebral bone, nerves within the brain tissue, tissue responsible for cardiac arrhythmia within the cardiac tissue, nerves along the bronchial tree, one or more esophageal branches of the vagus nerve, and one or more nerves surrounding the bladder.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited.

What is claimed is:

1. A tissue treatment catheter, comprising:
a catheter shaft having an outer wall sized and shaped for delivery through a radial artery to a blood vessel of a patient, the catheter shaft having a plurality of lumens extending longitudinally through the catheter shaft between a distal end and a proximal end, wherein the plurality of lumens includes a guidewire lumen within a guidewire lumen wall and a cable lumen, wherein the catheter shaft includes a stiffening web extending from the guidewire lumen wall, and wherein the plurality of lumens include a first fluid lumen defined between the outer wall, the guidewire lumen wall, and the stiffening web, and wherein an outer wall thickness of the outer wall is less than a stiffening wall thickness of the stiffening web;
an ultrasound transducer distally positioned relative to the distal end of the catheter shaft, the ultrasound transducer including a piezoelectric transducer body;
a balloon surrounding the ultrasound transducer, wherein the first fluid lumen is configured to provide a fluid to the balloon at a pressure and a flow rate sufficient to protect the ultrasound transducer and non-target tissue of the blood vessel from thermal injury; and
a single electrical cable extending through the cable lumen, the single electrical cable electrically connected to the ultrasound transducer, wherein the single electrical cable is configured to deliver sufficient electrical energy during sonication to the transducer such that the transducer thermally induces modulation of neural fibers surrounding the blood vessel sufficient to improve a measurable physiological parameter corresponding to a diagnosed condition of the patient.

2. The tissue treatment catheter of claim 1, wherein the stiffening wall thickness is at least twice the outer wall thickness.

3. The tissue treatment catheter of claim 2, wherein the outer wall thickness is in a range of 0.002 to 0.003 inch.

4. The tissue treatment catheter of claim 2, wherein the stiffening wall thickness is in a range of 0.003 to 0.007 inch.

5. The tissue treatment catheter of claim 1, wherein the cable lumen is within a cable lumen wall, and wherein the stiffening web extends between the guidewire lumen wall and the cable lumen wall.

6. The tissue treatment catheter of claim 1, wherein the plurality of lumens include a hybrid lumen having the guidewire lumen and the cable lumen, and wherein the stiffening web extends between the hybrid lumen and the outer wall.

7. The tissue treatment catheter of claim 1, wherein the plurality of lumens includes a second fluid lumen defined between the outer wall, the guidewire lumen wall, and the stiffening web.

8. The tissue treatment catheter of claim 7, wherein the first fluid lumen and the second fluid lumen are symmetric about a vertical plane extending through the stiffening web.

9. The tissue treatment catheter of claim 1, wherein a web surface of the stiffening web, a guidewire lumen surface of the guidewire lumen wall, and a cable lumen surface of a cable lumen wall containing the cable lumen face radially outward toward the first fluid lumen, and wherein an outer wall surface of the outer wall faces radially inward toward the first fluid lumen.

10. The tissue treatment catheter of claim 1, wherein an outer diameter of the catheter shaft has a French gauge of 5 or less.

11. The tissue treatment catheter of claim 1, wherein an outer diameter of the catheter shaft has a French gauge of 4 to 5, and wherein the first fluid lumen has a length in a range of 145 to 175 cm and is configured to deliver fluid at a head pressure of 34 psi or less.

12. The tissue treatment catheter of claim 1, wherein an outer diameter of the catheter shaft is 0.05 inch to 0.07 inch, and wherein the first fluid lumen has a length of at least 145 cm and a minimum cross-sectional area of 0.00055 square inch.

13. A tissue treatment system, comprising:
a controller including a signal generator; and
a treatment catheter including a catheter shaft having an outer wall sized and shaped for delivery through a radial artery to a blood vessel of a patient, the catheter shaft having a plurality of lumens extending longitudinally through the catheter shaft between a distal end and a proximal end, wherein the plurality of lumens includes a guidewire lumen within a guidewire lumen wall and a cable lumen, wherein the catheter shaft includes a stiffening web extending from the guidewire lumen wall, and wherein the plurality of lumens includes a first fluid lumen defined between the outer wall, the guidewire lumen wall, and the stiffening web, and wherein an outer wall thickness of the outer wall is less than a stiffening wall thickness of the stiffening web;
an ultrasound transducer distally positioned relative to the distal end of the catheter shaft, the ultrasound transducer including a piezoelectric transducer body;
a balloon surrounding the ultrasound transducer, wherein the first fluid lumen is configured to provide a cooling fluid to the balloon at a pressure and flow rate sufficient to protect the ultrasound transducer and non-target tissue of the blood vessel from thermal injury; and
a single electrical cable extending through the cable lumen, the single electrical cable electrically connecting the signal generator to the ultrasound transducer, wherein the single electrical cable is configured to deliver sufficient electrical energy during sonication to the transducer such that the transducer thermally induces modulation of neural fibers surrounding the blood vessel sufficient to improve a measurable physiological parameter corresponding to a diagnosed condition of the patient.

14. The tissue treatment system of claim 13, wherein the stiffening wall thickness is at least twice the outer wall thickness.

15. The tissue treatment system of claim 14, wherein the outer wall thickness is in a range of 0.002 to 0.003 inch.

16. The tissue treatment system of claim 14, wherein the stiffening wall thickness is in a range of 0.003 to 0.007 inch.

17. The tissue treatment system of claim 13, wherein the cable lumen is within a cable lumen wall, and wherein the stiffening web extends between the guidewire lumen wall and the cable lumen wall.

18. The tissue treatment system of claim 13, wherein the plurality of lumens include a hybrid lumen having the guidewire lumen and the cable lumen, and wherein the stiffening web extends between the hybrid lumen and the outer wall.

19. The tissue treatment system of claim 13, wherein the plurality of lumens includes a second fluid lumen defined between the outer wall, the guidewire lumen wall, and the stiffening web.

20. The tissue treatment system of claim 19, wherein the first fluid lumen and the second fluid lumen are symmetric about a vertical plane extending through the stiffening web.

21. The tissue treatment system of claim 13, wherein a web surface of the stiffening web, a guidewire lumen surface of the guidewire lumen wall, and a cable lumen surface of a cable lumen wall containing the cable lumen face radially outward toward the first fluid lumen, and wherein a shaft wall surface of the outer wall faces radially inward toward the first fluid lumen.

22. The tissue treatment system of claim 13, wherein an outer diameter of the catheter shaft has a French gauge of 5 or less.

23. A method, comprising:
advancing a treatment catheter through a radial artery to a blood vessel of a patient, the treatment catheter including a catheter shaft having an outer wall, and a plurality of lumens extending longitudinally through the catheter shaft between a distal end and a proximal end, wherein the plurality of lumens includes a guidewire lumen within a guidewire lumen wall and a cable lumen, wherein the catheter shaft includes a stiffening web extending from the guidewire lumen wall, and wherein the plurality of lumens includes a first fluid lumen defined between the outer wall, the guidewire lumen wall, and the stiffening web, and wherein an outer wall thickness of the outer wall is less than a stiffening wall thickness of the stiffening web, an ultrasound transducer distally positioned relative to the distal end of the catheter shaft, the ultrasound transducer including a piezoelectric transducer body, a balloon surrounding the ultrasound transducer, wherein the first fluid lumen is configured to provide a cooling fluid to the balloon at a pressure and flow rate sufficient to protect the ultrasound transducer and non-target tissue of the blood vessel from thermal injury, and a single electrical cable extending through the cable lumen, the single electrical cable electrically connected to the ultrasound transducer, wherein the single electrical cable is configured to deliver sufficient electrical energy during sonication to the transducer such that the transducer thermally induces modulation of neural fibers surrounding the blood vessel sufficient to improve a measurable physiological parameter corresponding to a diagnosed condition of the patient; and
applying a voltage to the ultrasound transducer, using the single electrical cable, to cause the piezoelectric transducer body to generate ultrasonic waves.

24. The method of claim 23, wherein the stiffening wall thickness is at least twice the outer wall thickness.

25. The method of claim 24, wherein the outer wall thickness is in a range of 0.002 to 0.003 inch.

26. The method of claim 24, wherein the stiffening wall thickness is in a range of 0.003 to 0.007 inch.

27. The method of claim 23, further comprising transferring cooling fluid between a reservoir and the balloon using the first fluid lumen to cool the ultrasound transducer and at least a portion of the single electrical cable that extends through the cable lumen.

28. The method of claim 23, further comprising ablating tissue of the blood vessel into which the ultrasound transducer is inserted, using the ultrasonic waves that are generated by the piezoelectric transducer body.
